(19) United States
(12) Patent Application Publication
(10) Pub. No.: US 2026/0069727 A1
(43) Pub. Date: Mar. 12, 2026

(54) FORMULATIONS FOR RADIOTHERAPY AND DIAGNOSTIC IMAGING AND USE THEREOF IN TREATMENT, DIAGNOSIS AND IMAGING DISEASES

(71) Applicant: Clarity Pharmaceuticals Ltd, Eveleigh (AU)

(72) Inventors: Lachlan Eion McInnes, New South Wales (AU); Michael David Ironside, New South Wales (AU)

(21) Appl. No.: 19/103,854

(22) PCT Filed: Aug. 16, 2023
(86) PCT No.: PCT/AU2023/050776

§ 371 Date: Feb. 14, 2025

(30) Foreign Application Priority Data
Aug. 16, 2022 (AU) 2022902326

(51) Int. Cl. A61K 51/08; A61K 9/08; A61K 47/02; A61K 47/10; A61K 47/12; A61K 47/22

(52) U.S. Cl. CPC A61K 51/088; A61K 9/08; A61K 47/02; A61K 47/10; A61K 47/12; A61K 47/22; A61K 2121/00; A61K 2123/00

(57) ABSTRACT

The present invention relates to formulations of radiolabelled compounds that are of use in radiotherapy and diagnostic imaging. Specifically, the present invention relates to aqueous formulations of 67 Cu radiolabelled compounds of Formula (I) further comprising a buffer, gentisic acid, ethanol and ascorbic acid for use in radiotherapy and diagnostic imaging.

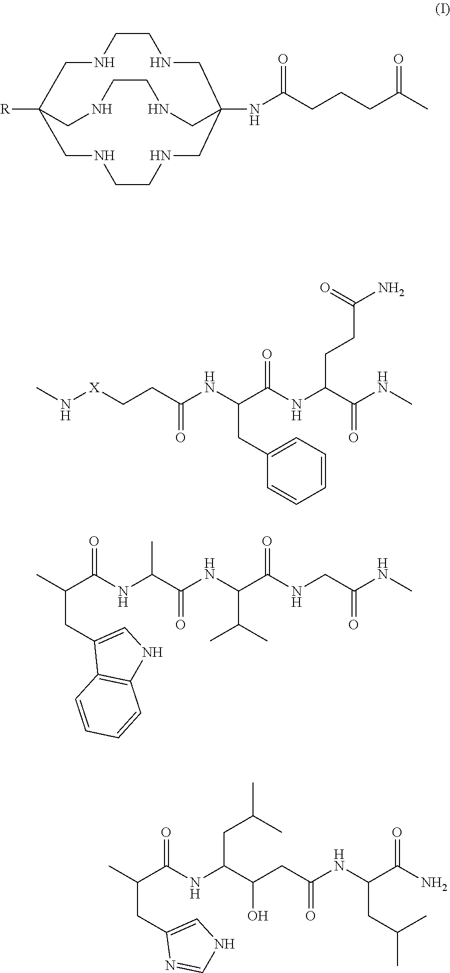

### FIELD

The present invention relates to formulations of radiolabelled compounds that are of use in radiotherapy and diagnostic imaging.

### BACKGROUND

Radiolabelled compounds or ligands may be used as radiopharmaceuticals in applications such as radiotherapy or diagnostic imaging. Of particular use, are radiolabelled compounds that show some propensity for selectively targeting a particular site in vivo, (for example, a particular receptor), and subsequently delivering the radioisotope to the desired site of action. This requires that the ligand comprises a component to complex the radioisotope and a further component to target the desired site.

One of the known problems associated with such a ligand is the premature dissociation of the radioisotope prior to the arrival of the ligand-radioisotope complex at the site of action. Not only does this reduce the efficacy of the complex, but the loss of the radioisotope to areas where radiotherapeutic effects are not intended, may result in adverse consequences.

Dissociation of the radioisotope from the ligand may occur as a result of transchelation, where the radioisotope transfers to another biological ligand in vivo. Again, this leads to a reduced therapeutic effect and also delivery of a radioisotope to areas where treatment is not required.

The ligand to be radiolabelled and the radioisotope are usually stored and transported to the patient in separate containers to minimise the above problems relating to dissociation prior to administration. The ligand may be transported as a lyophilized powder at reduced temperatures in order to prolong stability of the compound. The radioisotope can then be combined with the ligand to form the radiopharmaceutical, just prior to administration, which can serve to minimise dissociation of the radioisotope prior to the complex reaching the site of action.

Another problem associated with radiolabelled compounds is that the use of a radioisotope may result in radiolysis, or destruction or partial destruction of the ligand. As a radioisotope undergoes spontaneous decay and subsequent release of radiation, this energy may be sufficient to induce cleavage of bonds and cause subsequent destruction of the ligand. In addition to the reduced efficacy of the radiopharmaceutical, release of the radioisotope or release of fragments of the original drug product also occurs, resulting in the delivery of radiation to unwanted sites.

As many radiopharmaceuticals are designed to be administered parenterally, i.e. non-orally and usually as a solution, the ligand itself must be soluble in a pharmaceutically acceptable solvent or carrier. As is known in the art, the solubility of a particular compound in any given solvent may be unpredictable. Although the solubility of a particular compound in a particular solvent may be known, the solubility of an analogue of the compound in a different solvent system may be quite different. This then presents difficulties to one seeking to develop a formulation of a compound and especially a pharmaceutically acceptable injectable formulation.

Pharmaceutical formulations typically include one or more excipients that affect the compound in some way, such as the enhancement of solubility of the compound or increasing stability of the compound while in solution. Alternatively, additional excipients may be used to provide other features to the formulation, such as preservatives, buffers and the like.

While many thousands of formulations of ligand-radioisotope complexes have been documented, there is no expectation that the excipients used in such formulations would provide the required solubility, stability and bioavailability of any newly developed complex. Furthermore, one cannot expect that a particular combination of excipients would further prevent or minimise the dissociation of the radioisotope or minimise radiolysis from occurring. For example, a formulation comprising a particular combination of excipients may provide the required solubility, stability and bioavailability of a particular complex and be completely unsuitable for another complex. In addition, where stability may be found at a low level of radioactivity, it may not be found at high levels of radioactivity. This may be the difference in levels of activity used for a diagnostic dose in the order of a few hundred mega-becquerels, for example, versus a higher therapeutic dose of radioactivity in the order of several giga-becquerels. Or this difference may be in the levels of radioactivity when a large multidose batch is prepared and then subsequently aliquoted into smaller lower radioactivity doses. The differences in stability may also be derived by the type of radioactivity such as from gamma, beta or alpha emissions from one isotope to another being used in the same drug substance for diagnostic imaging in one instance, to delivering a therapeutic cell killing dose in another instance.

The degree of radiolysis of ligand-radioisotope complexes depends on the level of radioactivity, the level of specific activity, and the structure of the ligand. A formulation may provide the required stability for a complex with one radionuclide, but it may not be found with another. A case in point is positron-emitting copper-64 (⁶⁴Cu, t_{1/2}=12.7 h) and beta particle emitting copper-67 (⁶⁷Cu, t_{1/2}=2.58 d). Beta emission often results in complexes being less stable than their stably labelled counterparts, thus making complexes labelled with ⁶⁷Cu more prone to radiolysis. In view of this, formulations may vary widely depending on the radionuclide that is employed, for example, by addition of stabilising agents or increased amounts of stabilizing agents. As well as expense, increased levels of additional agents in the formulation may introduce new problems such as sedimentation in the formulation or pH imbalance of the formulation.

Accordingly, desirable formulations of ligand-radioisotope complexes need to be tailored in order to display the requisite stability in relation to radiolysis and dissociation of the radioisotope, while also being pharmaceutically acceptable. The present invention seeks to address these problems in relation to a specific ligand complex.

### SUMMARY

In one aspect of the present invention, there is provided an aqueous formulation comprising a compound of Formula (I), or a salt thereof, complexed with a ⁶⁷Cu ion:

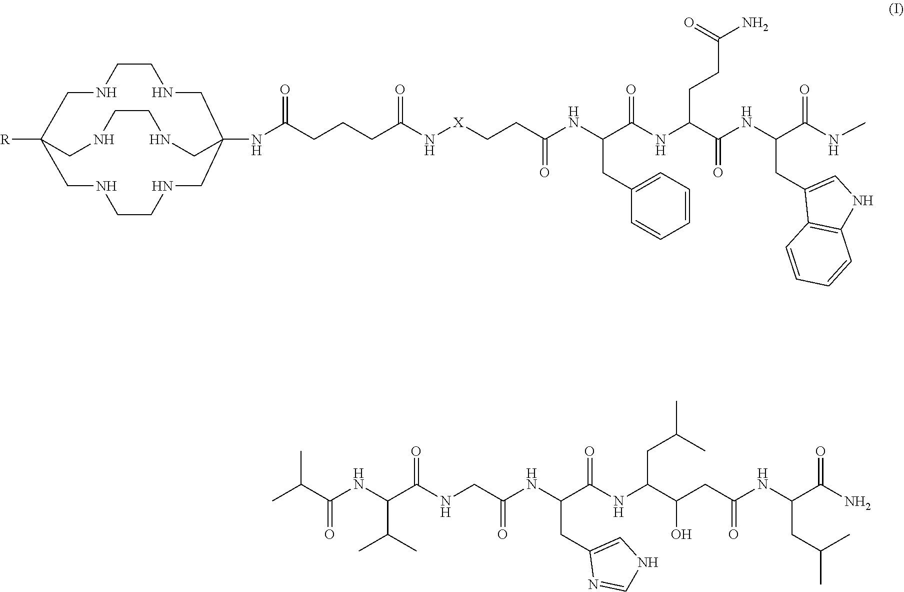

wherein:
X is

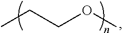

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A):

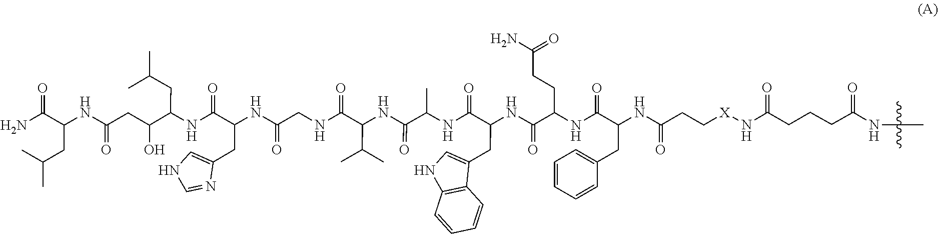

the formulation further comprising a buffer and:
about 0.01% to about 0.1% (w/v) gentisic acid or a salt thereof;
about 1% to about 7% (v/v) ethanol; and
about 4% to about 10% (w/v) ascorbic acid or a salt thereof.

In an embodiment of the first aspect, R is a group of the formula (A):

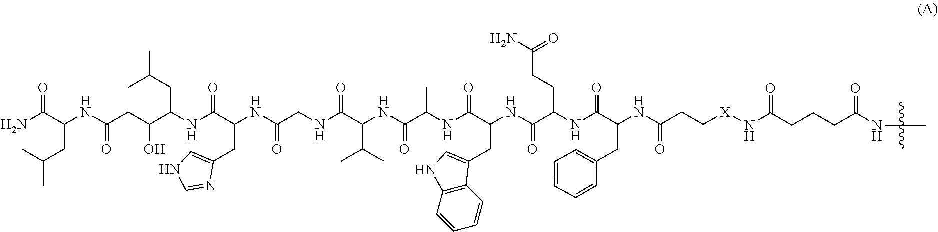

wherein X is as defined above, such as with the below stereochemistry:

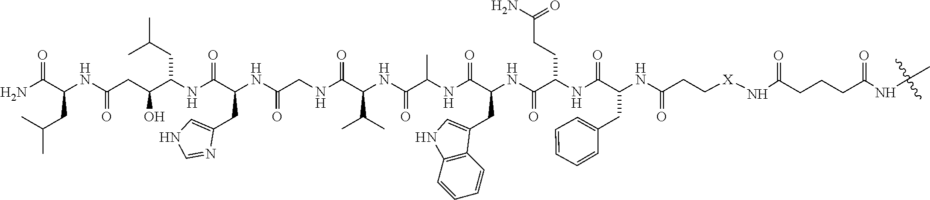

In an embodiment of the first aspect, R is an optionally substituted C₁-C₁₂ alkyl.

In a further embodiment, R is methyl.

In an embodiment of the first aspect, X is

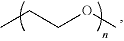

wherein n is an integer from 1 to 10.

In a further embodiment, X is

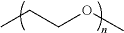

and n is 4.

In certain embodiments, the radiochemical purity of the formulation is more than about 90% for a time of at least 96 hours. In certain embodiments, the radiochemical purity of the formulation is more than about 92% for a time of at least 96 hours. In certain embodiments, the radiochemical purity of the formulation is more than about 94% for a time of at least 96 hours. In certain embodiments, the radiochemical purity of the formulation is more than about 96% for a time of at least 96 hours. In certain embodiments, the radiochemical purity of the formulation is more than about 98% for a time of at least 96 hours.

In some embodiments, the amount of free ⁶⁷Cu present in the formulation is not more than about 5%. In some embodiments, the amount of free ⁶⁷Cu present in the formulation is not more than about 4%. In other embodiments, the amount of free ⁶⁷Cu present in the formulation is less than about 1%. In other embodiments, the amount of free ⁶⁷Cu present in the formulation is less than about 2%.

In certain embodiments, the pH of the formulation is between about 4 and about 8. In other embodiments, the pH of the formulation is about 4, about 5, about 6, about 7 or about 8. In some embodiments, the pH of the formulation is about 6.

According to a further aspect of the present invention, there is provided a process for preparing an aqueous formulation comprising a compound of Formula (I), or a salt thereof, complexed with a ⁶⁷Cu ion, the method comprising the steps of:
i) dissolving a compound of Formula (I), or a salt thereof, in a buffer solution comprising gentisic acid, or a salt thereof;
ii) adding a solution of a ⁶⁷Cu ion to the solution of step i);
iii) filtering the solution obtained from step ii); and
iv) diluting the reaction by addition of aqueous ethanol and ascorbic acid;
   to recover an aqueous formulation comprising a compound of Formula (I), or a salt thereof, complexed with a ⁶⁷Cu ion.

According to another aspect of the present invention, there is provided an aqueous formulation prepared by a process as defined above.

The aqueous formulation of the present invention may also be prepared by providing certain components of the formulation as a kit of parts, where the kit comprises at least a compound of Formula (I), or a salt thereof, and the ⁶⁷Cu ion that is intended to be complexed with the compound of Formula (I), or the salt thereof, in which the compound of Formula (I), or a salt thereof, and the ⁶⁷Cu ion are provided separately in the kit and may be combined to form the aforementioned complex prior to administration.

Accordingly, in another aspect the present invention provides a kit for making an aqueous formulation comprising a compound of Formula (I), or a salt thereof, complexed with a ⁶⁷Cu ion, the kit comprising:
a container comprising a lyophilised compound of Formula (I):

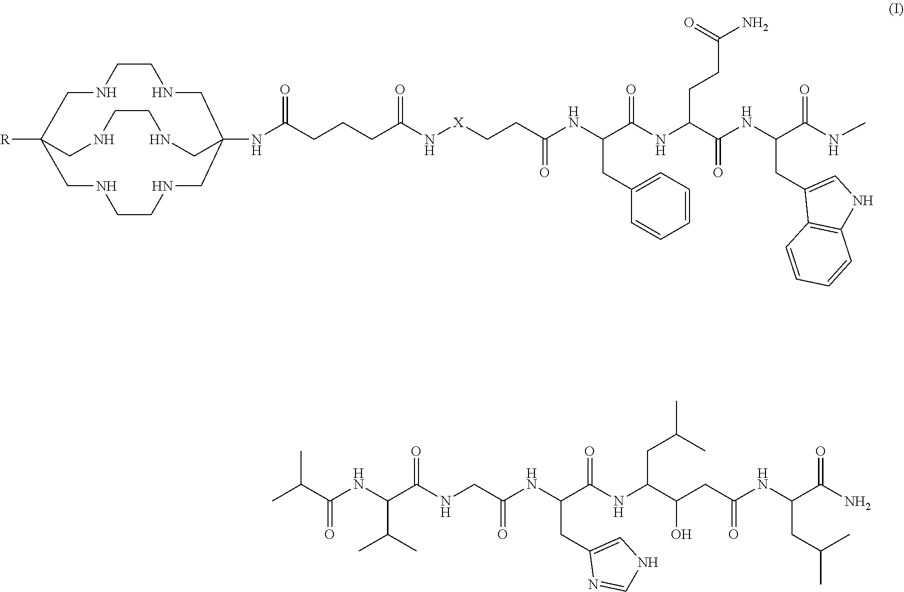

or a salt thereof, wherein:
X is

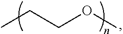

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A):

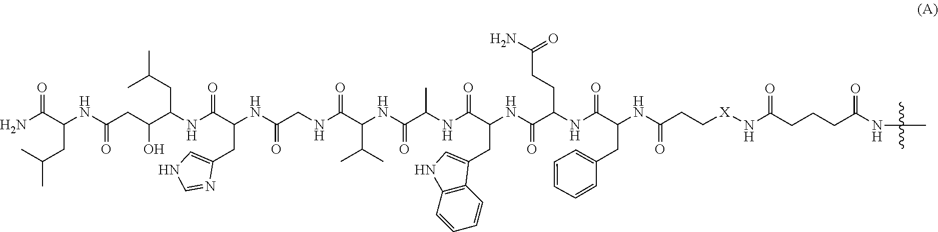

a container comprising a solution of a ⁶⁷Cu ion; and
instructions for preparing an aqueous formulation as defined in an earlier aspect, including the addition of sodium phosphate buffer, gentisic acid, or a salt thereof, ethanol and ascorbic acid, or a salt thereof.

A further aspect of the present invention provides a kit for making an aqueous formulation as defined in an earlier aspect for parenteral administration, the kit comprising: a container comprising a lyophilised compound of Formula (I):

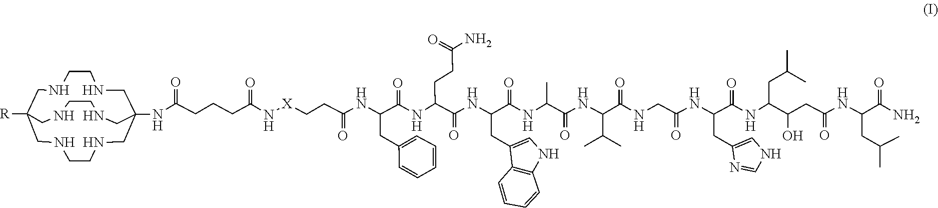

or a salt thereof, wherein:
X is

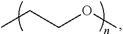

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A):

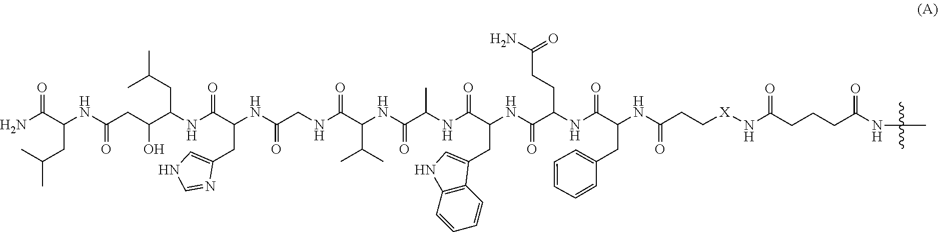

a container comprising a solution of a ⁶⁷Cu ion;
a container consisting sodium phosphate buffer and gentisic acid, or a salt thereof;
a container consisting of aqueous ethanol and ascorbic acid, or a salt thereof; and instructions for preparing an aqueous formulation as defined in an earlier aspect.

The radioactivity of the formulations disclosed herein is due to the presence of the ⁶⁷Cu radioisotope. In certain embodiments of the formulations disclosed herein, the radioactive concentration of the formulation as a result of the ⁶⁷Cu radioisotope is about 1.0 GBq/mL. In other embodiments, the radioactive concentration of the formulation is about 0.95 GBq/mL. In other embodiments, the radioactive concentration of the formulation is about 0.90 GBq/mL. In other embodiments, the radioactive concentration of the formulation is about 0.85 GBq/mL. In other embodiments, the radioactive concentration of the formulation is about 0.80 GBq/mL. In other embodiments, the radioactive concentration of the formulation is about 0.75 GBq/mL. In other embodiments, the radioactive concentration of the formulation is about 0.70 GBq/mL. In other embodiments, the radioactive concentration of the formulation is about 0.65 GBq/mL. In other embodiments, the radioactive concentration of the formulation is about 0.60 GBq/mL. In other embodiments, the radioactive concentration of the formulation is about 0.55 GBq/mL. In other embodiments, the radioactive concentration of the formulation is about 0.50 GBq/mL. In other embodiments, the radioactive concentration of the formulation is about 0.45 GBq/mL. In other embodiments, the radioactive concentration of the formulation is about 0.40 GBq/mL. In other embodiments, the radioactive concentration of the formulation is about 0.35 GBq/mL. In other embodiments, the radioactive concentration of the formulation is about 0.30 GBq/mL. In other embodiments, the radioactive concentration of the formulation is about 0.25 GBq/mL. In other embodiments, the radioactive concentration of the formulation is about 0.20 GBq/mL. In other embodiments, the radioactive concentration of the formulation is about 0.15 GBq/mL. In other embodiments, the radioactive concentration of the formulation is about 0.10 GBq/mL. Concentrations in a range of the values above are also contemplated as part of the present invention.

Another aspect of the present invention provides a method for radioimaging, diagnosing or treating a cancer, the method comprising administering to a subject in need thereof an aqueous formulation according to the first aspect.

According to another aspect, the present invention provides a method of treating a cancer in a subject, the method comprising administering to a subject in need thereof the aqueous formulation according to the first aspect.

According to a further aspect, the present invention provides a method of radioimaging a subject, the method comprising administering to a subject in need thereof the aqueous formulation according to the first aspect.

The methods disclosed herein comprise the step of administering a formulation of the present invention, wherein the formulation comprises a compound of Formula (I) complexed with a ⁶⁷Cu radioisotope. Administration of the complex of ⁶⁷Cu and the compound of Formula (I) results in the localisation of the compound at a site expressing a GRP receptor, such that the radioisotope undergoes decay and provides a therapeutic effect to the area of the subject at which the complex is bound. In some embodiments, the methods disclosed herein comprise the step of administering a dose of the formulation of the present invention having between about 1 GBq to about 20 GBq of radioactivity.

In certain embodiments, the methods disclosed herein comprise the step of administering a dose of the formulation of the present invention having about 20 GBq of radioactivity. In certain embodiments, the methods disclosed herein comprise the step of administering a dose of the formulation of the present invention having about 19 GBq of radioactivity. In certain embodiments, the methods disclosed herein comprise the step of administering a dose of the formulation of the present invention having about 18 GBq of radioactivity. In certain embodiments, the methods disclosed herein comprise the step of administering a dose of the formulation of the present invention having about 17 GBq of radioactivity. In certain embodiments, the methods disclosed herein comprise the step of administering a dose of the formulation of the present invention having about 16 GBq of radioactivity. In certain embodiments, the methods disclosed herein comprise the step of administering a dose of the formulation of the present invention having about 15 GBq of radioactivity. In certain embodiments, the methods disclosed herein comprise the step of administering a dose of the formulation of the present invention having about 14 GBq of radioactivity. In certain embodiments, the methods disclosed herein comprise the step of administering a dose of the formulation of the present invention having about 13 GBq of radioactivity. In other embodiments, the methods disclosed herein comprise the step of administering a dose of the formulation of the present invention have about 12 GBq of radioactivity. In other embodiments, the methods disclosed herein comprise the step of administering a dose of the formulation of the present invention have about 11 GBq of radioactivity. In other embodiments, the methods disclosed herein comprise the step of administering a dose of the formulation of the present invention have about 10 GBq of radioactivity. In certain embodiments, the methods disclosed herein comprise the step of administering a dose of the formulation of the present invention having about 9 GBq of radioactivity. In other embodiments, the methods disclosed herein comprise the step of administering a dose of the formulation of the present invention have about 8 GBq of radioactivity. In certain embodiments, the methods disclosed herein comprise the step of administering a dose of the formulation of the present invention having about 7 GBq of radioactivity. In certain embodiments, the methods disclosed herein comprise the step of administering a dose of the formulation of the present invention having about 6 GBq of radioactivity. In other embodiments, the methods disclosed herein comprise the step of administering a dose of the formulation of the present invention have about 5 GBq of radioactivity. In certain embodiments, the methods disclosed herein comprise the step of administering a dose of the formulation of the present invention having about 4 GBq of radioactivity. In other embodiments, the methods disclosed herein comprise the step of administering a dose of the formulation of the present invention have about 3 GBq of radioactivity. In certain embodiments, the methods disclosed herein comprise the step of administering a dose of the formulation of the present invention having about 2 GBq of radioactivity. In certain embodiments, the methods disclosed herein comprise the step of administering a dose of the formulation of the present invention having about 1 GBq of radioactivity. Doses in a range of the values above are also contemplated as part of the present invention.

The present inventors have found that the formulations disclosed herein containing a compound of Formula (I) complexed with ⁶⁷Cu allows for high doses of radioactivity to be delivered to a subject, where the formulation and complexed compound is stable and radioactively pure for at least 96 hours. Furthermore, the formulations disclosed herein allow for the delivery of a high dose of radioactivity in the form of ⁶⁷Cu as a result of the high concentration of the compound of Formula (I) complexed with ⁶⁷Cu. The present inventors have found that even though a high concentration of a radiolabelled compound is incorporated into the formulation, the compound of Formula (I) does not degrade by radiolysis even with the increased concentration and in the presence of the radioisotope in the formulation. Without wishing to be bound by theory, the present inventors believe that the formulations disclosed herein allow for the administration of high dose of radiation to be delivered as part of a complex of a compound of Formula (I), where the formulation also prevents degradation of the compound of Formula (I) by radiolysis even in such high concentrations and radioactivity. This is shown in FIGS. 1 to 4, which indicate the stability of the formulations over a time of up to 96 hours.

In certain embodiments, the methods disclosed herein relate to a cancer associated with expression of a gastrin-releasing peptide (GRP) receptor. In certain embodiments, the cancer is a tumour. In other embodiments, the tumour is associated with prostate cancer, breast cancer, ovarian cancer, urinary cancer, small cell lung cancers, glioblastoma or gastrointestinal stromal tumours.

In certain methods for the treatment of a cancer as disclosed herein, the method further comprises the radioimaging of the subject after the administration of a formulation of the present invention. In certain embodiments, radioimaging of the subject is by PET-CT or SPECT-CT.

### BRIEF DESCRIPTION OF THE FIGURES

The invention will herein be described by way of example only with reference to the following non-limiting Figures in which:

FIG. 1 is a chromatogram from the analysis by radioHPLC of a formulation comprising [⁶⁷Cu]Sar-BBN 12 hours after preparation of the formulation. This chromatogram shows the radiochemical purity of the formulation is 97.68%, indicating that the ⁶⁷Cu radioisotope present in the formulation is complexed with the compound of Formula (I).

FIG. 2 is a chromatogram from the analysis by radioHPLC of a formulation comprising [⁶⁷Cu]Sar-BBN 96 hours after preparation of the formulation. This chromatogram shows the radiochemical purity of the formulation at 93.96%, indicating that the ⁶⁷Cu radioisotope present in the formulation is complexed with the compound of Formula (I).

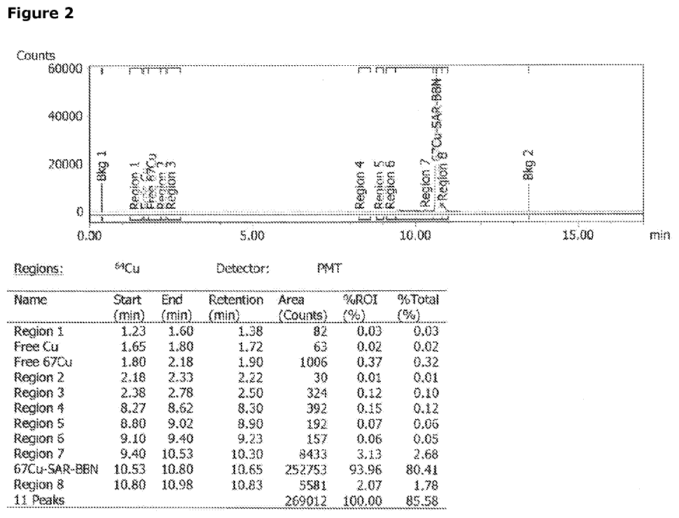

FIG. 3 is a chromatogram from the analysis by radioTLC of a formulation comprising [⁶⁷Cu]Sar-BBN 12 hours after preparation of the formulation. This chromatogram indicates that a large majority of the ⁶⁷Cu radioisotope present in the formulation is complexed with the compound of Formula (I) with very little ⁶⁷Cu detected.

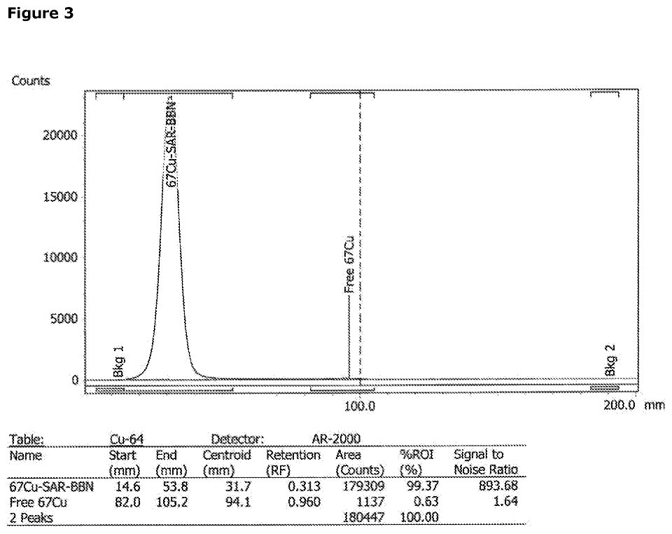

FIG. 4 is a chromatogram from the analysis by radioTLC of a formulation comprising [⁶⁷Cu]Sar-BBN 96 hours after preparation of the formulation. This chromatogram indicates that a large majority of the ⁶⁷Cu radioisotope present in the formulation is complexed with the compound of Formula (I) with very little free ⁶⁷Cu detected.

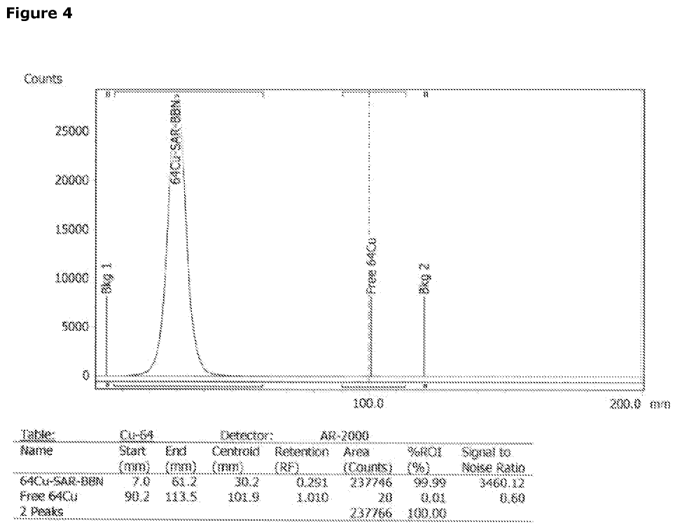

### DETAILED DESCRIPTION

The present invention relates to stable formulations of a specific radioisotope-ligand complex. The present inventors have found that the formulations of a complex disclosed herein minimise dissociation of the radioisotope from the ligand and/or minimise radiolysis of the ligand arising from the radioisotope.

The formulations of a radioisotope-ligand complex referred to herein are stable in solution and under physiological conditions for a time. The stability of the formulation relates to the stability of the complex, where the radioisotope may undergo dissociation or the complex may undergo radiolysis. The stability of the complex can be measured by considering the radiochemical purity of the formulation. Radiochemical purity is defined as the amount of the radioisotope complexed by the sarcophagine ligand expressed as a percentage of the total amount of the radioisotope present in the formulation. The radioisotope may be present in the formulation as a complex with the sarcophagine ligand, as a free radioisotope or as part of a radiolysis product. In certain embodiments, the radiochemical purity of the formulation is more than about 90% for a time of at least 96 hours. In other embodiments, the radiochemical purity of the formulation is more than about 91% for a time of at least 96 hours. In other embodiments, the radiochemical purity of the formulation is more than about 92% for a time of at least 96 hours. In other embodiments, the radiochemical purity of the formulation is more than about 93% for a time of at least 96 hours. In other embodiments, the radiochemical purity of the formulation is more than about 94% for a time of at least 96 hours. In other embodiments, the radiochemical purity of the formulation is more than about 95% for a time of at least 96 hours. In other embodiments, the radiochemical purity of the formulation is more than about 96% for a time of at least 96 hours. In other embodiments, the radiochemical purity of the formulation is more than about 97% for a time of at least 96 hours. In the embodiments, described herein, radiochemical purity may be determined by techniques such as radioHPLC, where the amount of the radioisotope in either its complexed or free form is determined and compared with the amount of the radioisotope introduced to the formulation. A person skilled in the art would understand the necessary conditions for separation, analysis and quantification of the formulation by radioHPLC. Nonetheless, in certain embodiments, the mobile phase used to analyse a formulation as disclosed herein by radioHPLC comprises about 0.1% trifluoroacetic acid (TFA) in water. In certain embodiments, the mobile phase used to analyse a formulation as disclosed herein by radioHPLC comprises about 0.1% trifluoroacetic acid (TFA) in acetonitrile.

The stability of the complex can also be measured by considering the amount of free copper, i.e. free copper radioisotope that is intended to be complexed with the sarcophagine ligand, present in the formulation. In certain embodiments, the amount of free copper radioisotope present in formulation is not more than about 5%. In other embodiments, the amount of free copper radioisotope present in the formulation is not more than about 4%. In other embodiments, the amount of free copper present in the formulation is not more than about 3%. In other embodiments, the amount of free copper present in the formulation is not more than about 2%. In other embodiments, the amount of free copper present in the formulation is not more than about 1%. In other embodiments, the amount of free ⁶⁷Cu present in the formulation is less than about 1%. In other embodiments, the amount of free ⁶⁷Cu present in the formulation is less than about 2%.

The stable formulations of the invention comprise a compound of Formula (I), or salts thereof:

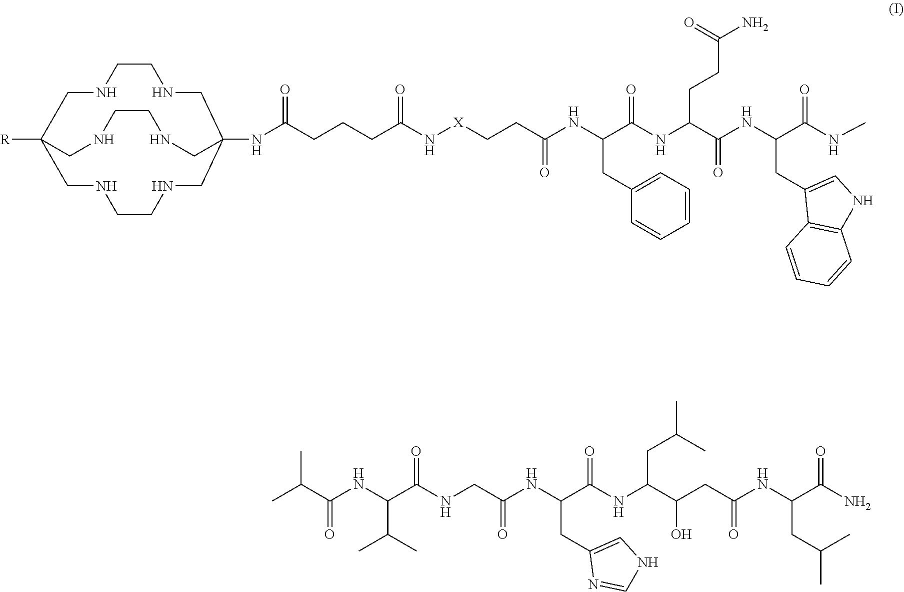

wherein X is

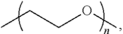

where n is an integer from 1 to 10;
R is a group selected from the group consisting of H, OH, halogen, cyano, NO₂, NH₂, optionally substituted C₁-C₁₂ alkyl, optionally substituted amino, optionally substituted amide, optionally substituted aryl and a group of the formula (A):

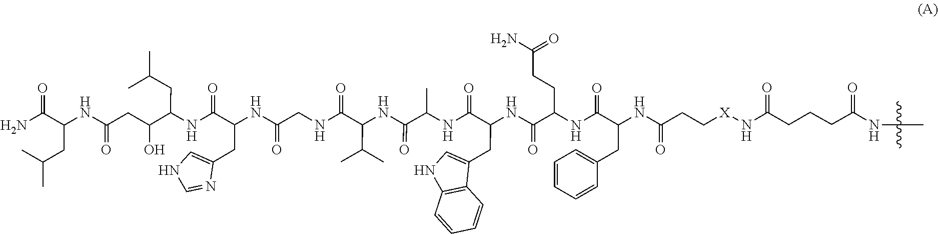

wherein X is a as defined above.

The compounds of Formula (I), or salts thereof, contain a peptide, where the peptide has the sequence D-Phe-Gln-Trp-Ala-Val-Gly-His-Sta-Leu-NH₂ and has the following structure:

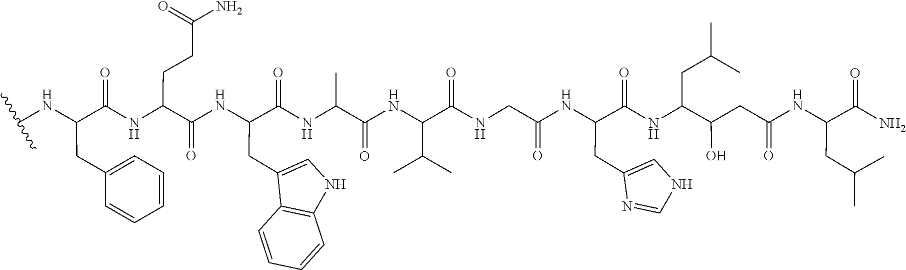 [SMILES: *NC(Cc1ccccc1)C(=O)NC(CCC(N)=O)C(=O)NC(Cc1c[nH]c2ccccc12)C(=O)NC(C)C(=O)NC(C(=O)NCC(=O)NC(Cc1c[nH]cn1)C(=O)NC(CC(C)C)C(O)CC(=O)NC(CC(C)C)C(N)=O)C(C)C]

The above peptide fragment is related to the family of bombesin receptor peptides that show antagonist (or agonist) activity at the gastrin-releasing peptide (GRP) receptor. The GRP receptor is known to be expressed or overexpressed on the membrane of various cancers and may be a target for diagnostic or therapeutic purposes. Compounds containing the bombesin-like peptide as depicted herein may bind to sites expressing the GRP receptor and where a suitable radionuclide is also delivered as part of the compound, a diagnostic or therapeutic effect may be provided locally. The amino acids of the bombesin-like peptide as used herein may have a specific stereochemistry, as depicted below:

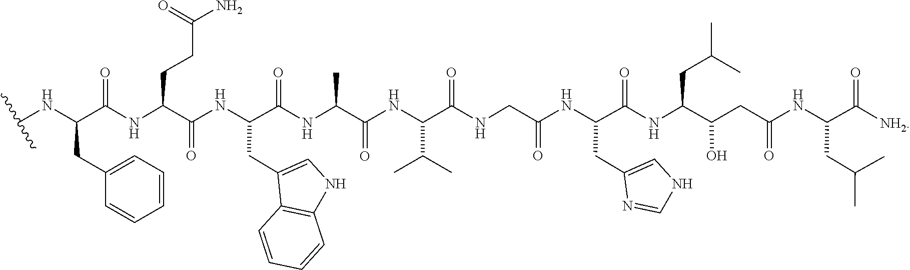

The compounds of Formula (I), or salts thereof, also contain a nitrogen-containing macrocycle, which is capable of chelating metal ions. The macrocycle of Formula (I) is a 3,6,10,13,16,19-hexaazabicyclo[6.6.0]icosane and may be referred to as a “sarcophagine”. The sarcophagine of Formula (I) contain six nitrogen atoms, where one or more of the nitrogen atoms may be protected with a suitable protecting group.

The compounds of Formula (I), or salts thereof, contain a sarcophagine and a bombesin-like peptide, where the peptide is bound to terminal position of the sarcophagine via a linker group. As depicted herein, the linker group comprises a propylamide group bound directly to the terminal position of the sarcophagine. The propylamide group is then attached to a linker comprising a polyethylene glycol (PEG) group, having between 1 and 10 repeat units. The PEG group has the following structure:

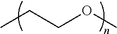

where n is an integer from 1 to 10.

The present inventors have found that compounds of Formula (I), or salts thereof, containing the combination of a sarcophagine and a bombesin-like peptide, or peptide that serves as either an agonist or antagonist for the gastrin-releasing peptide receptor, for instance, where the sarcophagine and the bombesin-like peptide are bound together via a propylamide group (adjacent to the sarcophagine) and the linker comprising the PEG group are capable of chelating a metal ion and binding to a target receptor. Without wishing to be bound by theory, the present inventors believe that it is the combination of the sarcophagine, bombesin-like peptide, propylamide group and the linker comprising a PEG group that provides the specific advantages that are observed and discussed below. Although the properties of the compounds of the present invention are as a result of each component of the compound, the present inventors believe that the presence of the linker comprising a PEG group to modify biodistribution, metabolism and excretion profiles, increases the overall biocompatibility of the compounds and may be responsible for the observed advantages.

In certain embodiments, the group R in the compound of Formula (I), or the salt thereof, is selected from the group consisting of H, OH, halogen, cyano, NO₂, NH₂, optionally substituted C₁-C₁₂ alkyl, optionally substituted amino, optionally substituted C₁-C₁₂ amide, optionally substituted C₆-C₁₀ aryl and a group of the formula (A):

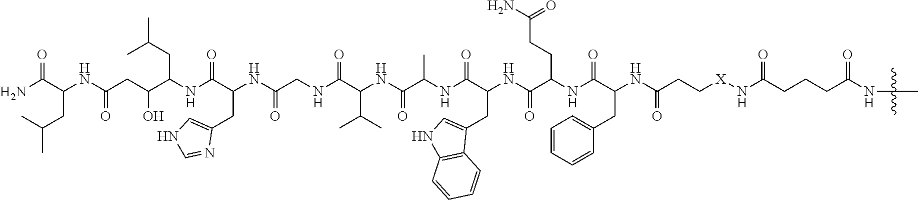

wherein X is as defined above.

In certain embodiments, R is a group of the formula (A) having the stereochemistry as defined below:

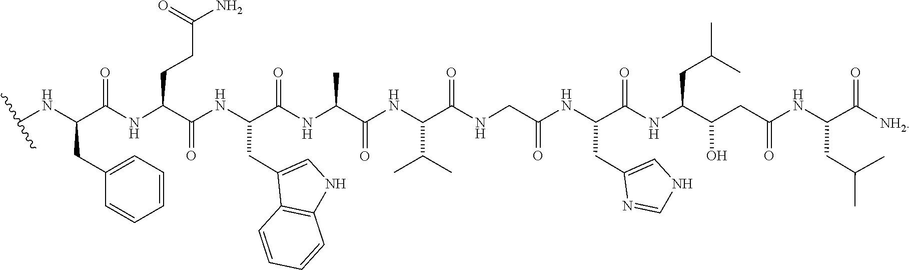

In certain embodiments, R is an optionally substituted C₁-C₁₂ alkyl group. In an embodiment, R is an optionally substituted C₁ alkyl group. In another embodiment, R is an optionally substituted methyl group. In another embodiment, R is an unsubstituted C₁-C₁₂ alkyl group. In another embodiment, R is an unsubstituted C₁ alkyl group. In another embodiment, R is an unsubstituted methyl group.

In certain embodiments, the compound of Formula (I), or the salt thereof, has the following structure:

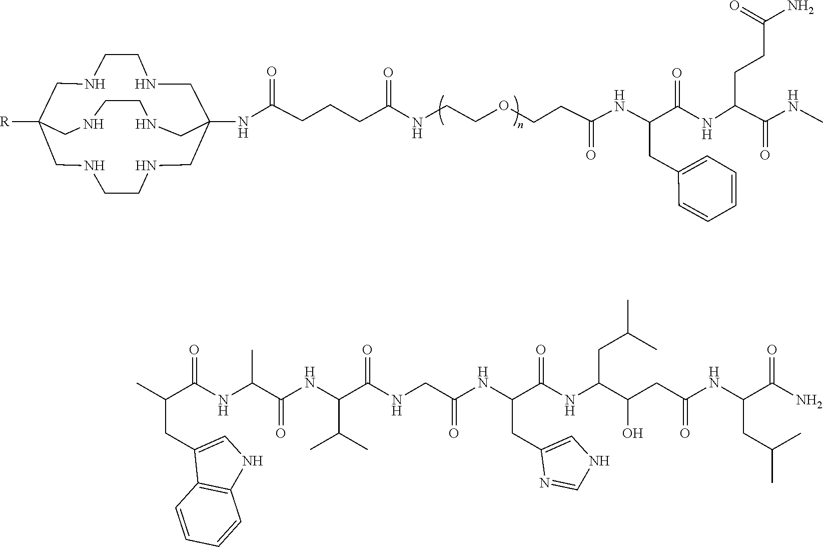

wherein n is an integer from 1 to 10.

In a specific embodiment, the compound of Formula (I), or the salt thereof, has the following structure:

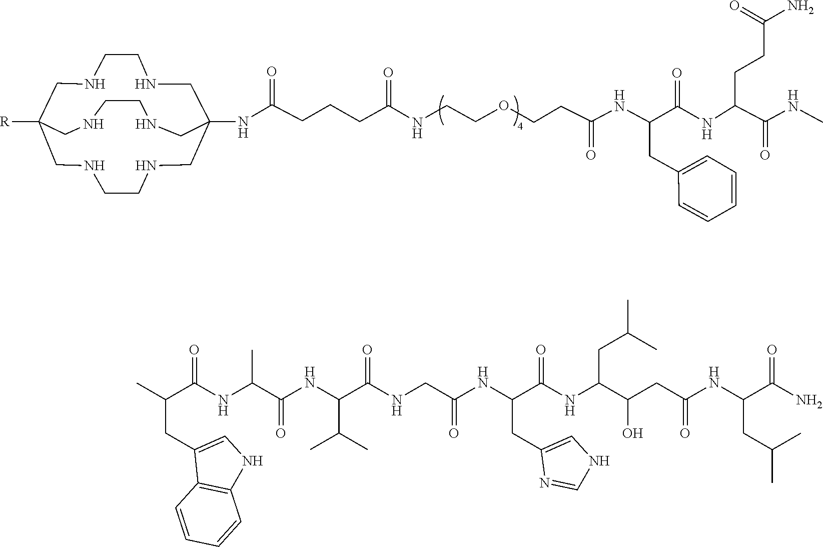

In certain embodiments, R is an optionally substituted C₁-C₁₂ amide group. In an embodiment, R is an optionally substituted C₁ amide group. In an embodiment, R is a C₁ amide group that is further substituted by one or more groups.

In certain embodiments, R is a group of the formula (A):

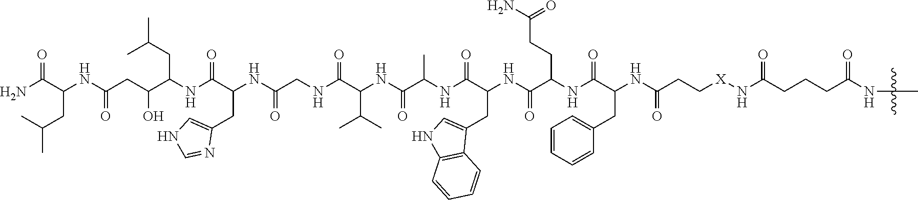

wherein X is as defined above.

In an embodiment, R is a group of the formula (A) and X is a group of the formula

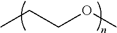

and n is an integer from 1 to 10.

In an embodiment, R is a group of the formula (A), X is a group of the formula

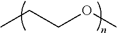

and n is 4.

In an embodiment, R is an unsubstituted methyl group and X is a group of the formula

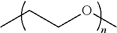

and n is an integer from 1 to 10.

In an embodiment, R is an unsubstituted methyl group, X is a group of the formula

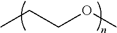

and n is 4.

In certain embodiments, the compound of Formula (I) has the following structure of Formula (Ia):

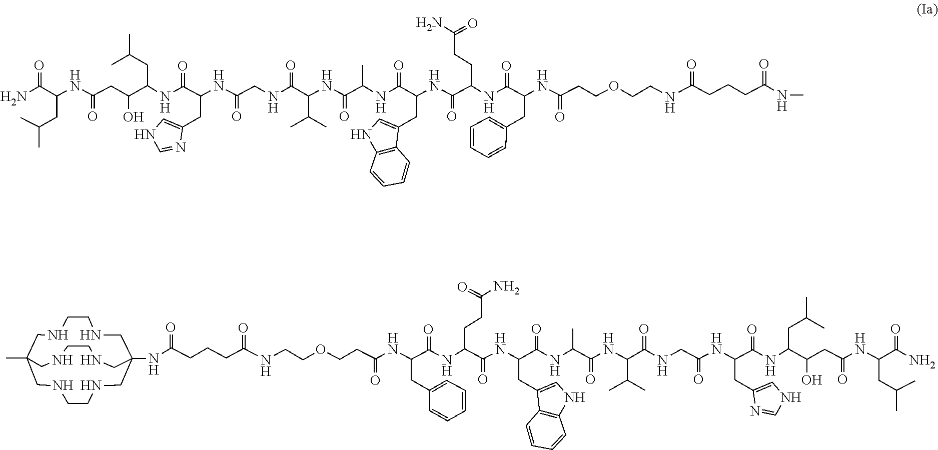

In other embodiments, the compound of Formula (I), or the salt thereof, has the structure of Formula (Ia), where the stereochemistry is defined as below:

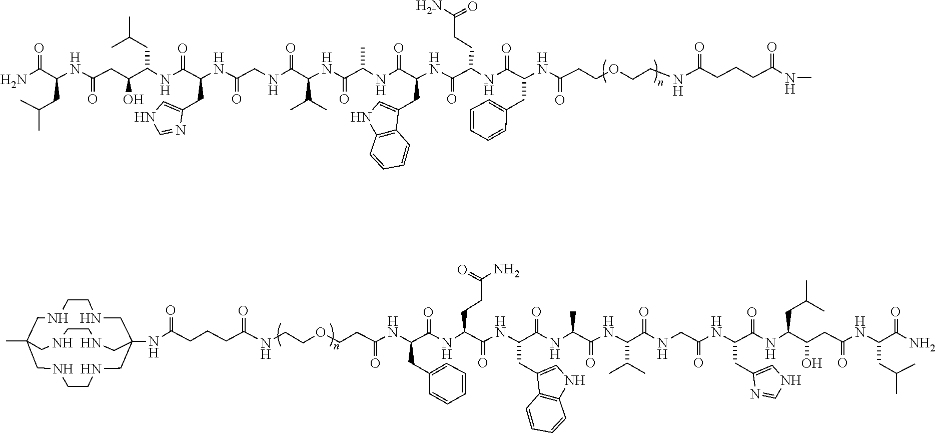

In a further embodiment, the compound of formula (I), or the salt thereof, has the structure of Formula (Ib):

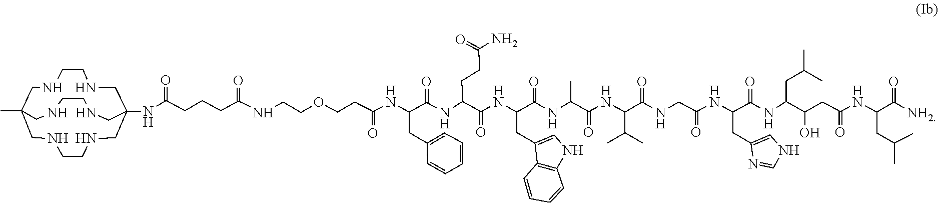 [SMILES: CC(C)CC(NC(=O)CC(O)C(CC(C)C)NC(=O)C(Cc1c[nH]cn1)NC(=O)CNC(=O)C(NC(=O)C(C)NC(=O)C(Cc1c[nH]c2ccccc12)NC(=O)C(CCC(N)=O)NC(=O)C(Cc1ccccc1)NC(=O)CCOCCNC(=O)CCCC(=O)NC12CNCCNCC(C)(CNCCNC1)CNCCNC2)C(C)C)C(N)=O]

In other embodiments, the compound of Formula (I), or the salt thereof, has the structure of Formula (Ib), where the stereochemistry is defined as below:

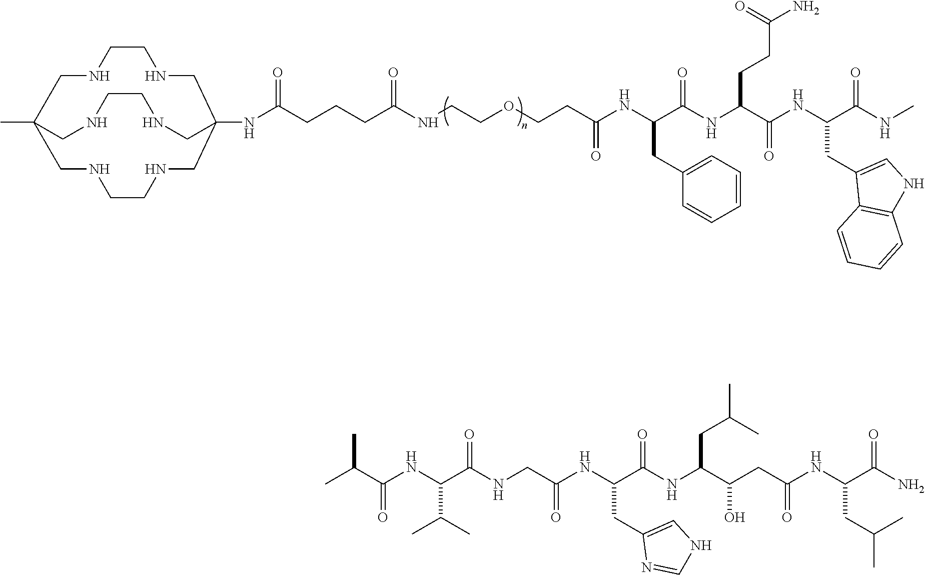

As used herein, the term “alkyl” refers to a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like.

As used herein, the term “amide” refers to a functional group consisting of a carbonyl group attached to a nitrogen atom. Therefore, the term “optionally substituted amide” refers to an amide functional group that bears further substitution.

As used herein, the term “aryl” refers to a group or part of a group that denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. Typically an aryl group is a C₆-C₁₈ aryl group.

As used herein, the term “cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₉ cycloalkyl group.

As used herein, the term “halogen” represents chlorine, fluorine, bromine or iodine.

As used herein, the term “heteroalkyl” refers to a straight- or branched-chain alkyl group preferably having from 2 to 12 carbons, more preferably 2 to 6 carbons in the chain, in which one or more of the carbon atoms (and any associated hydrogen atoms) are each independently replaced by a heteroatomic group selected from S, O, P and NR′ where R′ is selected from the group consisting of H, optionally substituted C₁-C₁₂alkyl, optionally substituted C₃-C₁₂cycloalkyl, optionally substituted C₆-C₁₈aryl, and optionally substituted C₁-C₁₈heteroaryl. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl

As used herein, the term “heteroaryl” either alone or as part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl, 1-, 2-, or 3-indolyl, and 2-, or 3-thienyl. A heteroaryl group is typically a C₁-C₁₈ heteroaryl group.

As used herein, the term “C₁-C₁₂ alkylene” refers to a bivalent straight or branched chain aliphatic hydrocarbon group, where the group has 1 to 12 carbon atoms in the chain.

As used herein, the term “optionally substituted” used in connection with a particular group indicates that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R^{b}, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl, wherein R^{a}, R^{b}, R^{c} and R^{d} are each independently selected from the group consisting of H, C₁-C₁₂alkyl, C₁-C₁₂haloalkyl, C₂-C₁₂alkenyl, C₂-C₁₂alkynyl, C₂-C₁₀ heteroalkyl, C₃-C₁₂cycloalkyl, C₃-C₁₂cycloalkenyl, C₂-C₁₂heterocycloalkyl, C₂-C₁₂ heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments, each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, OH, OCH₃, CF₃, OCF₃, NO₂, NH₂, COOH, COOCH₃ and CN.

As used herein, the term “salt” refers to acid addition salts and base addition salts of the compound, where the salt is prepared from an inorganic or organic acid, or an inorganic or organic base. In some embodiments, the salts of the compounds of the present invention may be pharmaceutically acceptable salts.

As used herein, the term “pharmaceutically acceptable salts” refers to salts that retain the desired biological activity of the above-identified compounds and may also be acid addition salts or base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “complex” refers to a compound that is then coordinated by a metal ion, such as a compound of Formula (I), or a salt thereof, coordinated with ⁶⁷Cu.

The administrable formulations of the present invention comprise a complex of a compound of Formula (I), or a salt thereof, and the radioisotope ⁶⁷Cu. The radioisotope, may also be referred to as a radionuclide. The ligand of the present specification has been found to be particularly successful in complexing copper ions, especially Cu²⁺ ions. A person skilled in the art would also appreciate that a complex of a compound of Formula (I), or a salt thereof, and a radioisotope may be achieved by contacting the compound of Formula (I), or a salt thereof, with the radioisotope that is to be complexed, such that the compound of Formula (I), or a salt thereof, is complexed with the radioisotope. This may involve the mixing of the compound of Formula (I), or a salt thereof, and the radioisotope in a suitable solvent system (such as that specifically described herein).

In certain embodiments, the formulations disclosed herein comprise a buffer solution. In some embodiments, the buffer solution is a phosphate buffer solution, meaning that the formulation comprises both phosphate ions and copper ions in solution. The present inventors understand that copper phosphate salts form readily and subsequently precipitate, which is unwanted for any pharmaceutically acceptable formulations and products. The formulations disclosed herein comprise a copper radioisotope, which provides the radiotherapeutic effect. In certain embodiments, the formulations disclosed herein comprise a buffer solution and in specific embodiments, the buffer solution is a phosphate buffer solution, which is known to be a physiologically acceptable component. Even though it is known that a formulation comprising both a copper ion and phosphate ions have a known potential to form insoluble precipitates, the present inventors have found unexpectedly that the formulations comprising both of these components as disclosed herein do not result in the unwanted (and detrimental) precipitation of copper salts. The present inventors also understand that the formation of metal salts, e.g. a copper phosphate salt, is also dependent on the pH of the formulation and that avoiding or preventing the unwanted formation of metal salts requires careful consideration of factors such as pH, temperature, concentration of ions in solution and the like. With the knowledge that phosphate salts of copper can form at physiologically relevant pH ranges, the present inventors have unexpectedly found that the formulations disclosed herein comprising both a copper ion and phosphate ions in solution at physiological pH do not form copper phosphate salts and are therefore pharmaceutically acceptable for administration to a subject.

In some embodiments, the formulation is an aqueous formulation and the pharmaceutically acceptable carrier is a saline solution that includes a phosphate buffer. In a preferred embodiment, the pharmaceutically acceptable carrier is sodium phosphate buffer.

The formulations of the present invention are for administration to a subject in need thereof and therefore are of a pharmaceutically acceptable and physiologically acceptable pH in order to ensure stability of the components of the formulation and safety of the subject. In certain embodiments, the formulations of the present invention have a pH of between about 4 and about 8. In some embodiments, the pH of the formulation is about 4. In other embodiments, the pH of the formulation is about 5. In other embodiments, the pH of the formulation is about 6. In other embodiments, the pH of the formulation is about 7. In other embodiments, the pH of the formulation is about 8. In some embodiments, the pH of the formulation may be a range between pH values disclosed herein. In some embodiments, the pH of the formulation results from the chemical nature of the components present in the formulation. In other embodiments, the pH of the formulation results from the use of one or more buffers present in the formulation. In certain embodiments, the formulations disclosed herein comprise a buffer solution that maintains the formulation at a specific pH or range thereof. In some embodiments, the buffer solution present in the formulation not only contributes to determining and maintaining the pH of the formulation, but also assists in ensuring the stability of one or more components of the formulation. In some embodiments, the one or more components responsible for ensuring the pH of formulation may also stabilise the formulation and prevent radiolysis of the complex comprising the radioisotope.

In an embodiment, the aqueous formulation of the present invention comprises one or more stabilizers in a total amount of about 4% to about 12% (w/v). In an embodiment, the aqueous formulation of the present invention comprises one or more stabilizers in a total amount of about 4% (w/v). In another embodiment, the aqueous formulation of the present invention comprises one or more stabilizers in a total amount of about 4.5% (w/v). In another embodiment, the aqueous formulation of the present invention comprises one or more stabilizers in a total amount of about 5% (w/v). In another embodiment, the aqueous formulation of the present invention comprises one or more stabilizers in a total amount of about 5.5% (w/v). In another embodiment, the aqueous formulation of the present invention comprises one or more stabilizers in a total amount of about 6% (w/v). In another embodiment, the aqueous formulation of the present invention comprises one or more stabilizers in a total amount of about 6.5% (w/v). In another embodiment, the aqueous formulation of the present invention comprises one or more stabilizers in a total amount of about 7% (w/v). In another embodiment, the aqueous formulation of the present invention comprises one or more stabilizers in a total amount of about 7.5% (w/v). In another embodiment, the aqueous formulation of the present invention comprises one or more stabilizers in a total amount of about 8% (w/v). In another embodiment, the aqueous formulation of the present invention comprises one or more stabilizers in a total amount of about 8.5% (w/v). In another embodiment, the aqueous formulation of the present invention comprises one or more stabilizers in a total amount of about 9% (w/v). In another embodiment, a formulation of the present invention comprises one or more stabilizers in a total amount of about 9.5% (w/v). In another embodiment, the aqueous formulation of the present invention comprises one or more stabilizers in a total amount of about 10% (w/v). In another embodiment, the aqueous formulation of the present invention comprises one or more stabilizers in a total amount of about 10.5% (w/v). In another embodiment, the aqueous formulation of the present invention comprises one or more stabilizers in a total amount of about 11% (w/v). In another embodiment, the aqueous formulation of the present invention comprises one or more stabilizers in a total amount of about 11.5% (w/v). In another embodiment, the aqueous formulation of the present invention comprises one or more stabilizers in a total amount of about 12% (w/v). In other embodiments, the present invention also contemplates one or more stabilizers present in ranges between the aforementioned amounts.

In an embodiment, the aqueous formulation comprising a compound of Formula (I), or a salt thereof, complexed with ⁶⁷Cu comprises gentisic acid, or a salt thereof, as a stabilizer. Gentisic acid is also known as 2,5-dihydroxybenzoic acid, 5-hydroxysalicylic acid or hydroquinonecarboxylic acid. Salts of gentisic acid may include the sodium salt and the sodium salt hydrate. Any reference to gentisic acid may include a reference to salts thereof, where relevant. As discussed above, the pH of the formulations disclosed herein is between about 4 and about 8. Since the pKa (i.e. the acid dissociation constant) of gentisic acid is about 2.5, gentisic acid present in the formulations disclosed herein will be present in its dissociated form, i.e. as gentisate. Without wishing to be bound by theory, the present inventors believe that the use of either the free acid (i.e. gentisic acid) or a corresponding does not lead to any material difference in the formulation.

In an embodiment, gentisic acid, or a salt thereof, is present in the formulation in an amount of about 0.01% to about 0.1% (w/v). In an embodiment, gentisic acid, or a salt thereof, is present in the formulation in an amount of about 0.01% (w/v). In an embodiment, gentisic acid, or a salt thereof, is present in the formulation in an amount of about 0.015% (w/v). In an embodiment, gentisic acid, or a salt thereof, is present in the formulation in an amount of about 0.02% (w/v). In another embodiment, gentisic acid, or a salt thereof, is present in the formulation in an amount of about 0.025% (w/v). In another embodiment, gentisic acid, or a salt thereof, is present in the formulation in an amount of about 0.03% (w/v). In another embodiment, gentisic acid, or a salt thereof, is present in the formulation in an amount of about 0.035% (w/v). In another embodiment, gentisic acid, or a salt thereof, is present in the formulation in an amount of about 0.04% (w/v). In another embodiment, gentisic acid, or a salt thereof, is present in the formulation in an amount of about 0.045% (w/v). In another embodiment, gentisic acid, or a salt thereof, is present in the formulation in an amount of about 0.05% (w/v). In another embodiment, gentisic acid, or a salt thereof, is present in the formulation in an amount of about 0.055% (w/v). In another embodiment, gentisic acid, or a salt thereof, is present in the formulation in an amount of about 0.6% (w/v). In another embodiment, gentisic acid, or a salt thereof, is present in the formulation in an amount of about 0.065% (w/v). In another embodiment, gentisic acid, or a salt thereof, is present in the formulation in an amount of about 0.07% (w/v). In another embodiment, gentisic acid, or a salt thereof, is present in the formulation in an amount of about 0.075% (w/v). In another embodiment, gentisic acid, or a salt thereof, is present in the formulation in an amount of about 0.08% (w/v). In another embodiment, gentisic acid, or a salt thereof, is present in the formulation in an amount of about 0.085% (w/v). In another embodiment, gentisic acid, or a salt thereof, is present in the formulation in an amount of about 0.09% (w/v). In another embodiment, gentisic acid, or a salt thereof, is present in the formulation in an amount of about 0.095% (w/v). In another embodiment, gentisic acid, or a salt thereof, is present in the formulation in an amount of about 0.1% (w/v). In other embodiments, the present invention also contemplates gentisic acid, or a salt thereof, in ranges between the aforementioned amounts. In a preferred embodiment, gentisic acid, or a salt thereof, is present in the formulation in an amount of about 0.03 to about 0.04% (w/v).

Ascorbic acid, or a salt thereof, is also present as a stabilizer in the aqueous formulation. Ascorbic acid is also known as L-ascorbic acid or Vitamin C. Salts of ascorbic acid include sodium ascorbate, calcium ascorbate, potassium ascorbate and sodium ascorbyl phosphate. Derivatives of ascorbic acid are also contemplated. These include fatty acid esters of ascorbic acid, such as the palmitate ester of ascorbic acid, i.e. ascorbyl palmitate.

In an embodiment, ascorbic acid, or a salt thereof is present in an amount of about 4.0% to about 10.0% (w/v). In an embodiment, ascorbic acid, or a salt thereof, is present in the formulation in an amount of about 4.0% (w/v). In another embodiment, ascorbic acid, or a salt thereof, is present in the formulation in an amount of about 4.5% (w/v). In another embodiment, ascorbic acid, or a salt thereof, is present in the formulation in an amount of about 5.0% (w/v). In another embodiment, ascorbic acid, or a salt thereof, is present in the formulation in an amount of about 5.5% (w/v). In another embodiment, ascorbic acid, or a salt thereof, is present in the formulation in an amount of about 6.0% (w/v). In another embodiment, ascorbic acid, or a salt thereof, is present in the formulation in an amount of about 6.5% (w/v). In another embodiment, ascorbic acid, or a salt thereof, is present in the formulation in an amount of about 7.0% (w/v). In another embodiment, ascorbic acid, or a salt thereof, is present in the formulation in an amount of about 7.5% (w/v). In another embodiment, ascorbic acid, or a salt thereof, is present in the formulation in an amount of about 8.0% (w/v). In another embodiment, ascorbic acid, or a salt thereof, is present in the formulation in an amount of about 8.5% (w/v). In another embodiment, ascorbic acid, or a salt thereof, is present in the formulation in an amount of about 9.0% (w/v). In another embodiment, ascorbic acid, or a salt thereof, is present in the formulation in an amount of about 9.5% (w/v). In another embodiment, ascorbic acid, or a salt thereof, is present in the formulation in an amount of about 10.0% (w/v). In other embodiments, the present invention also contemplates ascorbic acid, or a salt thereof, in ranges between the aforementioned amounts. In a preferred embodiment, ascorbic acid, or a salt thereof, is present in the formulation in an amount of about 6.5% to about 8% (w/v).

L-methionine, or a salt thereof, may also be utilised as a stabiliser. The term L-methionine as used herein refers to the amino acid bearing an S-methyl thioether side chain. The addition of L-methionine to a formulation of the present invention may further enhance the stability of the formulation by preventing or minimising radiolysis of a radiolabelled complex of Formula (I), thereby increasing the radiochemical purity of the formulation.

The aqueous formulations of the present invention also comprise ethanol as a component. The ethanol used in the formulation may be anhydrous ethanol. Alternatively, the ethanol used in the aqueous formulation may not have been subject to drying processes and may be hydrated. In certain embodiments, the ethanol is aqueous ethanol. The ethanol is preferably pharmaceutical grade ethanol. The ethanol present in the formulation may further assist in preventing radiolysis of the radiolabelled complex of Formula (I).

In an embodiment, ethanol is present in the aqueous formulation in an amount of about 1% to about 7% (v/v). In an embodiment, ethanol is present in the formulation in an amount of about 1% (v/v). In another embodiment, ethanol is present in the formulation in an amount of about 1.5% (v/v). In another embodiment, ethanol is present in the formulation in an amount of about 2% (v/v). In another embodiment, ethanol is present in the formulation in an amount of about 2.5% (v/v). In another embodiment, ethanol is present in the formulation in an amount of about 3% (v/v). In another embodiment, ethanol is present in the formulation in an amount of about 3.5% (v/v). In another embodiment, ethanol is present in the formulation in an amount of about 4% (v/v). In another embodiment, ethanol is present in the formulation in an amount of about 4.5% (v/v). In another embodiment, ethanol is present in the formulation in an amount of about 5% (v/v). In another embodiment, ethanol is present in the formulation in an amount of about 5.5% (v/v). In another embodiment, ethanol is present in the formulation in an amount of about 6% (v/v). In another embodiment, ethanol is present in the formulation in an amount of about 6.5% (v/v). In another embodiment, ethanol is present in the formulation in an amount of about 7% (v/v). In a preferred embodiment, ethanol is present in the formulation in an amount of about 4% (v/v). In other embodiments, the present invention also contemplates ethanol in ranges between the aforementioned amounts.

The formulations of the present invention may also comprise sodium chloride as a component. The sodium chloride in the formulations of the present invention may be provided as a saline solution. A saline solution is defined as an aqueous solution of sodium chloride. For example, normal saline is defined as an aqueous solution of sodium chloride at a concentration of 0.9% (w/v). In an embodiment of the present invention, the sodium chloride of a formulation is provided by a saline solution.

The formulations of the present invention have a pH of about 4 to about 8. A person skilled in the art would understand that the pH of the formulation is an inherent characteristic of the formulation, attributed to the combination of the compound of Formula (I) or a complex thereof, and the remaining excipients of the formulation. The present inventors have found that this pH range provides for optimal radiolabelling efficiency.

In an embodiment, the pH of the formulation is from about 4 to about 8. In an embodiment, the pH of the formulation is about 4. In another embodiment, the pH of the formulation is about 4.5. In another embodiment, the pH of the formulation is about 5.0. In an embodiment, the pH of the formulation is about 5.5. In another embodiment, the pH of the formulation is about 5.6. In another embodiment, the pH of the formulation is about 5.7. In another embodiment, the pH of the formulation is about 5.8. In another embodiment, the pH of the formulation is about 5.9. In another embodiment, the pH of the formulation is about 6.0. In another embodiment, the pH of the formulation is about 6.1. In another embodiment, the pH of the formulation is about 6.2. In another embodiment, the pH of the formulation is about 6.3. In another embodiment, the pH of the formulation is about 6.4. In another embodiment, the pH of the formulation is about 6.5. In another embodiment, the pH of the formulation is about 7.0. In another embodiment, the pH of the formulation is about 7.5. In another embodiment, the pH of the formulation is about 8.0. In a preferred embodiment, the pH of the formulation is about 6.0.

Without wishing to be bound by theory, the present inventors believe that the stability of the formulations disclosed herein comprising a radiolabelled complex of a compound of Formula (I), gentisic acid or a salt thereof, ethanol and ascorbic acid or a salt thereof are a result of the combination of these components and the omission of one or more of these components results in a formulation having much lower stability. Furthermore, the present inventors also believe that the use of each of the listed components also results in the prevention and minimisation of any radiolysis of the radiolabelled complex, with the components of the formulation preventing the disintegration of the ligand containing the ⁶⁷Cu radioisotope. This in turn provides a radiotherapeutic compound, i.e. the compound of Formula (I) complexed with ⁶⁷Cu that remains intact and is therefore therapeutically effective for a longer time, thus increasing the efficiency of a given dose administered to the subject. This also means that the costs involved in preparing and treating a subject with a formulation disclosed herein is reduced, since more of the therapeutically active compound is effective and available for treatment over a longer amount of time.

In an embodiment, the aqueous formulation of the present invention comprises a compound of Formula (I), or a salt thereof, complexed with a ⁶⁷Cu ion, about 4% (v/v) ethanol, about 6.5 to about 8% (w/v) ascorbic acid and about 0.03 to about 0.04% (w/v) gentisic acid, or a salt thereof, wherein the formulation has a pH of about 6.0. In an embodiment, the aqueous formulation of the present invention comprises a compound of Formula (I), or a salt thereof, complexed with a ⁶⁷Cu ion, about 4% (v/v) ethanol, about 6.5 to about 8% (w/v) ascorbic acid and not more than 0.06% (w/v) gentisic acid, or a salt thereof, wherein the formulation has a pH of about 6.0. In a further embodiment, the aqueous formulation of the present invention comprises a compound of Formula (I), or a salt thereof, complexed with a ⁶⁷Cu ion, about 4% ethanol, about 6.5 to about 8% ascorbic acid and about 0.035% (w/v) gentisic acid, or a salt thereof, wherein the formulation has a pH of about 6.0.

Accordingly, the present invention also provides an aqueous formulation comprising a compound of Formula (I), or a salt thereof, complexed with a ⁶⁷Cu ion:

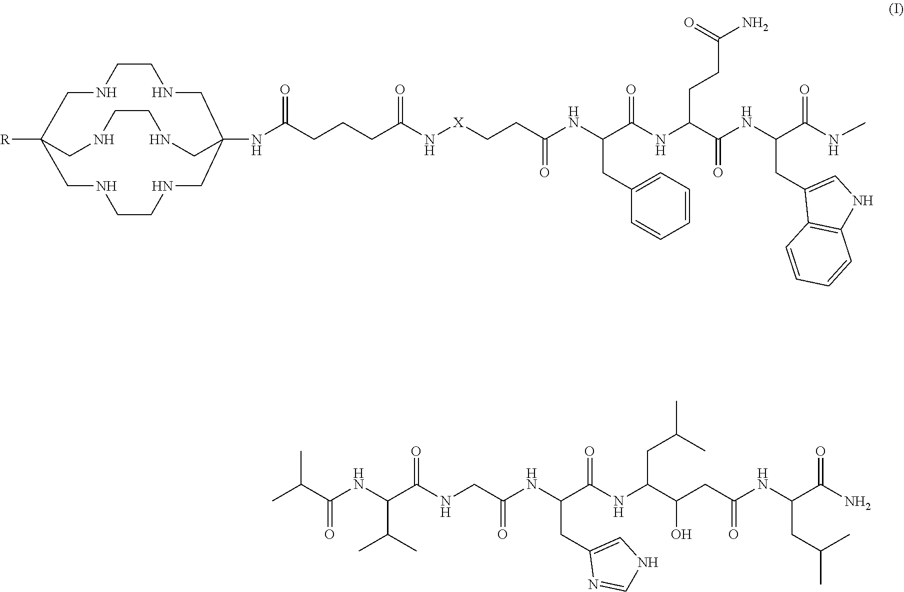

wherein:
X is

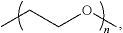

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A):

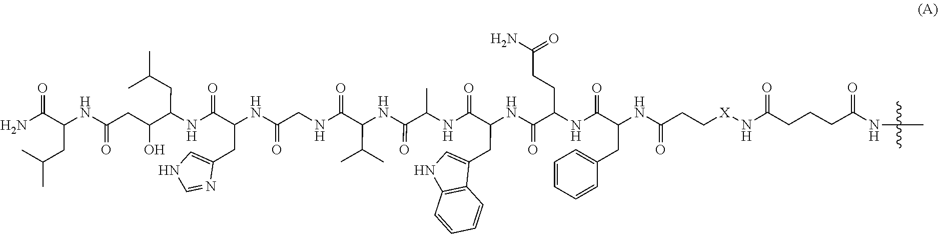

the formulation further comprising a buffer and:
about 1% to about 7% (v/v) ethanol;
about 4% to about 10% (w/v) ascorbic acid or a salt thereof; and
about 0.01% to about 0.1% (w/v) gentisic acid or a salt thereof;
wherein the formulation has a pH of between about 4 to about 8.

According to the present invention, a formulation of a complex of ⁶⁷Cu and a compound of Formula (I), or a salt thereof, may have a radiochemical purity of at least 90% for a time of at least 96 hours. This means that at least about 90% of the ⁶⁷Cu radioisotope present in the formulation is complexed with the compound of Formula (I), or a salt thereof, for at least 96 hours after preparation of the formulation. Where the ⁶⁷Cu radioisotope present in the formulation is not complexed with the compound of Formula (I), or a salt thereof, the ⁶⁷Cu radioisotope may be present as a free ⁶⁷Cu ion, or as part of a radiolysis product.

In an embodiment, the radiochemical purity of a formulation of the present invention comprising a complex of ⁶⁷Cu and a compound of Formula (I), or a salt thereof is about 90% at a time of about 96 hours after preparation of the formulation. In another embodiment, the radiochemical purity of a formulation of the present invention comprising a complex of ⁶⁷Cu and a compound of Formula (I), or a salt thereof is about 91% at a time of about 96 hours after preparation of the formulation. In another embodiment, the radiochemical purity of a formulation of the present invention comprising a complex of ⁶⁷Cu and a compound of Formula (I), or a salt thereof is about 92% at a time of about 96 hours after preparation of the formulation. In another embodiment, the radiochemical purity of a formulation of the present invention comprising a complex of ⁶⁷Cu and a compound of Formula (I), or a salt thereof is about 93% at a time of about 96 hours after preparation of the formulation. In another embodiment, the radiochemical purity of a formulation of the present invention comprising a complex of ⁶⁷Cu and a compound of Formula (I), or a salt thereof is about 94% at a time of about 96 hours after preparation of the formulation. In another embodiment, the radiochemical purity of a formulation of the present invention comprising a complex of ⁶⁷Cu and a compound of Formula (I), or a salt thereof is about 95% at a time of about 96 hours after preparation of the formulation. In another embodiment, the radiochemical purity of a formulation of the present invention comprising a complex of ⁶⁷Cu and a compound of Formula (I), or a salt thereof is about 96% at a time of about 96 hours after preparation of the formulation. In another embodiment, the radiochemical purity of a formulation of the present invention comprising a complex of ⁶⁷Cu and a compound of Formula (I), or a salt thereof is about 97% at a time of about 96 hours after preparation of the formulation. In another embodiment, the radiochemical purity of a formulation of the present invention comprising a complex of ⁶⁷Cu and a compound of Formula (I), or a salt thereof is about 98% at a time of about 96 hours after preparation of the formulation. In another embodiment, the radiochemical purity of a formulation of the present invention comprising a complex of ⁶⁷Cu and a compound of Formula (I), or a salt thereof is about 99% at a time of about 96 hours after preparation of the formulation.

In an embodiment, the radiochemical purity of a formulation of the present invention comprising a complex of ⁶⁷Cu and a compound of Formula (I), or a salt thereof is about 94% immediately after preparation of the formulation. In another embodiment, the radiochemical purity of a formulation of the present invention comprising a complex of ⁶⁷Cu and a compound of Formula (I), or a salt thereof is about 94% after about 1 h after preparation of the formulation. In another embodiment, the radiochemical purity of a formulation of the present invention comprising a complex of ⁶⁷Cu and a compound of Formula (I), or a salt thereof is about 94% after about 3 h after preparation of the formulation. In another embodiment, the radiochemical purity of a formulation of the present invention comprising a complex of ⁶⁷Cu and a compound of Formula (I), or a salt thereof is about 94% after about 6 h after preparation of the formulation. In another embodiment, the radiochemical purity of a formulation of the present invention comprising a complex of ⁶⁷Cu and a compound of Formula (I), or a salt thereof is about 94% after about 9 h after preparation of the formulation. In another embodiment, the radiochemical purity of a formulation of the present invention comprising a complex of ⁶⁷Cu and a compound of Formula (I), or a salt thereof is about 94% after about 12 h after preparation of the formulation. In another embodiment, the radiochemical purity of a formulation of the present invention comprising a complex of ⁶⁷Cu and a compound of Formula (I), or a salt thereof is about 94% after about 15 h after preparation of the formulation. In another embodiment, the radiochemical purity of a formulation of the present invention comprising a complex of ⁶⁷Cu and a compound of Formula (I), or a salt thereof is about 94% after about 18 h after preparation of the formulation. In another embodiment, the radiochemical purity of a formulation of the present invention comprising a complex of ⁶⁷Cu and a compound of Formula (I), or a salt thereof is about 94% after about 21 h after preparation of the formulation. In another embodiment, the radiochemical purity of a formulation of the present invention comprising a complex of ⁶⁷Cu and a compound of Formula (I), or a salt thereof is about 94% after about 24 h after preparation of the formulation. In another embodiment, the radiochemical purity of a formulation of the present invention comprising a complex of ⁶⁷Cu and a compound of Formula (I), or a salt thereof is about 94% after about 48 h after preparation of the formulation. In another embodiment, the radiochemical purity of a formulation of the present invention comprising a complex of ⁶⁷Cu and a compound of Formula (I), or a salt thereof is about 94% after about 72 h after preparation of the formulation.

### Preparation of an Aqueous Formulation of the Present Invention

The compound of Formula (I), or a salt thereof, complexed with a ⁶⁷Cu ion may be provided by mixing a compound of Formula (I), or a salt thereof, with a solution of a ⁶⁷Cu ion in the presence of a buffer and one or more stabilizing agents. The solution may then be filtered and the reaction subsequently diluted to provide the formulation comprising a compound of Formula (I), or a salt thereof, complexed with a ⁶⁷Cu ion. In one embodiment, the stabilizing agent is gentisic acid, or a salt thereof. In one embodiment, the reaction between the compound of Formula (I) and the ⁶⁷Cu ion is diluted with an aqueous ethanol solution comprising ascorbic acid, or a salt thereof.

Accordingly, the present invention provides a process for preparing an aqueous formulation comprising a compound of Formula (I) complexed with a ⁶⁷Cu ion, the method comprising the steps of:
i) dissolving a compound of Formula (I), or a salt thereof, in a buffer solution comprising gentisic acid, or a salt thereof;
ii) adding a solution of a ⁶⁷Cu ion to the solution of step i);
iii) filtering the solution obtained from step ii); and
iv) diluting the reaction by addition of aqueous ethanol and ascorbic acid;
   to recover an aqueous formulation comprising a compound of Formula (I), or a salt thereof, complexed with a ⁶⁷Cu ion.

The buffer may be a solution of ammonium acetate. Alternatively, the buffer solution may be a solution of sodium acetate. In a preferred embodiment, the buffer solution is a sodium phosphate buffer.

In certain embodiments, the buffer solution comprises a phosphate ion. In some embodiments, the buffer solution comprises a sodium phosphate salt. In other embodiments, the buffer solution comprises one or more sodium phosphate salts. In some embodiments, the buffer solution comprises two or more sodium phosphate salts. In some embodiments, the buffer solution comprises sodium phosphate dibasic. In other embodiments, the buffer solution comprises sodium phosphate monobasic. In some embodiments, the buffer solution comprises sodium phosphate dibasic and another phosphate salt. In some embodiments, the buffer solution comprises sodium phosphate monobasic and another phosphate salt. In other embodiments, the buffer solution comprises sodium phosphate dibasic and sodium phosphate monobasic. In some embodiments, the pH of the buffer solution is about 6. In other embodiments, the pH of the buffer solution is about 6.5. In other embodiments, the pH of the buffer solution is about 7.

The present inventors understand that the combination of a copper cation and a phosphate anion will typically lead to the formation of insoluble phosphate salts of copper. The present inventors have unexpectedly shown that in a process for preparing a formulation as defined herein, a ⁶⁷Cu radioisotope (which is present as a Cu²⁺ cation) is introduced to a solution comprising a phosphate buffer, yet no insolubility and formation of salts is observed. Without wishing to be bound by theory, the present inventors believe that the combination of at least several features (e.g. temperature, concentration and the like) have been found that allow for successful incorporation of the ⁶⁷Cu radioisotope into the compound of Formula (I).

The buffer solution also comprises gentisic acid, or a salt thereof, as a component. As previously described, salts of gentisic acid may include the sodium salt or the sodium salt hydrate. Other salts of gentisic acid are also contemplated. The buffer solution may comprise sodium gentisate at a concentration of between about 0.01 to about 0.1% (w/v). In an embodiment, the buffer solution comprises sodium gentisate at a concentration of about 0.01% (w/v). In another embodiment, the buffer solution comprises sodium gentisate at a concentration of about 0.015% (w/v). In another embodiment, the buffer solution comprises sodium gentisate at a concentration of about 0.02% (w/v). In another embodiment, the buffer solution comprises sodium gentisate at a concentration of about 0.025% (w/v). In another embodiment, the buffer solution comprises sodium gentisate at a concentration of about 0.03% (w/v). In another embodiment, the buffer solution comprises sodium gentisate at a concentration of about 0.035% (w/v). In another embodiment, the buffer solution comprises sodium gentisate at a concentration of about 0.04% (w/v). In another embodiment, the buffer solution comprises sodium gentisate at a concentration of about 0.045% (w/v). In another embodiment, the buffer solution comprises sodium gentisate at a concentration of about 0.05% (w/v). In another embodiment, the buffer solution comprises sodium gentisate at a concentration of about 0.055% (w/v). In another embodiment, the buffer solution comprises sodium gentisate at a concentration of about 0.06% (w/v). In another embodiment, the buffer solution comprises sodium gentisate at a concentration of about 0.065% (w/v). In another embodiment, the buffer solution comprises sodium gentisate at a concentration of about 0.07% (w/v). In another embodiment, the buffer solution comprises sodium gentisate at a concentration of about 0.075% (w/v). In another embodiment, the buffer solution comprises sodium gentisate at a concentration of about 0.08% (w/v). In another embodiment, the buffer solution comprises sodium gentisate at a concentration of about 0.085% (w/v). In another embodiment, the buffer solution comprises sodium gentisate at a concentration of about 0.095% (w/v). In another embodiment, the buffer solution comprises sodium gentisate at a concentration of about 0.1% (w/v). In a preferred embodiment, the buffering solution comprises sodium gentisate at a concentration of about 0.035% to 0.04% (w/v).

The reaction between the compound of Formula (I) and the ⁶⁷Cu ion is then diluted with an aqueous ethanol solution. As previously described, the ethanol may be anhydrous or may be previously subjected to drying procedures known in the art. The solution may comprise ethanol at a concentration of between about 1 to about 7% (v/v). In an embodiment, the solution comprises ethanol at a concentration of about 1% (v/v). In another embodiment, the solution comprises ethanol at a concentration of about 1.5% (v/v). In another embodiment, the solution comprises ethanol at a concentration of about 2% (v/v). In another embodiment, the solution comprises ethanol at a concentration of about 2.5% (v/v). In another embodiment, the solution comprises ethanol at a concentration of about 3% (v/v). In another embodiment, the solution comprises ethanol at a concentration of about 3.5% (v/v). In another embodiment, the solution comprises ethanol at a concentration of about 4% (v/v). In another embodiment, the solution comprises ethanol at a concentration of about 4.5% (v/v). In another embodiment, the solution comprises ethanol at a concentration of about 5% (v/v). In another embodiment, the solution comprises ethanol at a concentration of about 5.5% (v/v). In another embodiment, the solution comprises ethanol at a concentration of about 6% (v/v). In another embodiment, the buffering solution comprises ethanol at a concentration of about 6.5% (v/v). In another embodiment, the buffering solution comprises ethanol at a concentration of about 7% (v/v). In a preferred embodiment, the buffering solution comprises ethanol at a concentration of about 4% (v/v).

As mentioned above, the aqueous ethanol solution further comprises ascorbic acid, or a salt thereof. Ascorbic acid is also known as L-ascorbic acid or Vitamin C. Salts of ascorbic acid include sodium ascorbate, calcium ascorbate, potassium ascorbate and sodium ascorbyl phosphate. Derivatives of ascorbic acid are also contemplated. These include fatty acid esters of ascorbic acid, such as the palmitate ester of ascorbic acid, i.e. ascorbyl palmitate. In an embodiment, ascorbic acid, or a salt thereof is present in an amount of about 4.0% to about 10.0% (w/v). In an embodiment, ascorbic acid, or a salt thereof, is present in the solution in an amount of about 4.0% (w/v). In another embodiment, ascorbic acid, or a salt thereof, is present in the solution in an amount of about 4.5% (w/v). In another embodiment, ascorbic acid, or a salt thereof, is present in the solution in an amount of about 5.0% (w/v). In another embodiment, ascorbic acid, or a salt thereof, is present in the solution in an amount of about 5.5% (w/v). In another embodiment, ascorbic acid, or a salt thereof, is present in the solution in an amount of about 6.0% (w/v). In another embodiment, ascorbic acid, or a salt thereof, is present in the solution in an amount of about 6.5% (w/v). In another embodiment, ascorbic acid, or a salt thereof, is present in the solution in an amount of about 7.0% (w/v). In another embodiment, ascorbic acid, or a salt thereof, is present in the solution in an amount of about 7.5% (w/v). In another embodiment, ascorbic acid, or a salt thereof, is present in the solution in an amount of about 8.0% (w/v). In another embodiment, ascorbic acid, or a salt thereof, is present in the solution in an amount of about 8.5% (w/v). In another embodiment, ascorbic acid, or a salt thereof, is present in the solution in an amount of about 9.0% (w/v). In another embodiment, ascorbic acid, or a salt thereof, is present in the solution in an amount of about 9.5% (w/v). In another embodiment, ascorbic acid, or a salt thereof, is present in the solution in an amount of about 10.0% (w/v). In a preferred embodiment, ascorbic acid, or a salt thereof, is present in the solution in an amount of about 6.5% to about 8% (w/v).

According to an embodiment of the present invention, a compound of Formula (I), or a salt thereof, is mixed in a sodium phosphate buffer solution comprising gentisic acid, or a salt thereof. The compound of Formula (I) or a salt thereof, may be obtained as a solid. In an embodiment, the compound of Formula (I) or a salt thereof, is obtained as a lyophilised powder. In an embodiment, the compound of Formula (I) or a salt thereof, obtained as a lyophilised powder is mixed with a sodium phosphate buffer solution comprising gentisic acid or a salt thereof. In an embodiment, about 80 μg to about 160 μg of the compound of Formula (I) or a salt thereof, as a lyophilised powder is mixed with a sodium phosphate buffer solution comprising gentisic acid or a salt thereof.

A solution of a ⁶⁷Cu ion is added to the mixture of a compound of Formula (I), or a salt thereof, and the sodium phosphate buffer solution comprising gentisic acid or a salt thereof, and is allowed to stand for a time.

In an embodiment, the solution of a ⁶⁷Cu ion is a solution of a ⁶⁷Cu salt. In another embodiment, the solution of a ⁶⁷Cu ion is a solution of a chloride salt containing copper. In another embodiment, the solution of ⁶⁷Cu ion is a solution of [⁶⁷Cu]CuCl₂.

The solution of a ⁶⁷Cu ion is provided as an aqueous solution. The ⁶⁷Cu ion may be provided in an aqueous solution of hydrochloric acid. In an embodiment, the ⁶⁷Cu ion is provided in a solution of between about 0.01 to about 0.1 mol/L hydrochloric acid. In an embodiment, the ⁶⁷Cu ion is provided in a solution of about 0.01 mol/L hydrochloric acid. In another embodiment, the ⁶⁷Cu ion is provided in a solution of about 0.02 mol/L hydrochloric acid. In another embodiment, the ⁶⁷Cu ion is provided in a solution of about 0.05 mol/L hydrochloric acid. In another embodiment, the ⁶⁷Cu ion is provided in a solution of about 0.075 mol/L hydrochloric acid. In another embodiment, the ⁶⁷Cu ion is provided in a solution of about 0.1 mol/L hydrochloric acid. In a preferred embodiment, the ⁶⁷Cu ion is provided as [⁶⁷Cu]CuCl₂ in a solution of about 0.05 mol/L hydrochloric acid.

The solution of a ⁶⁷Cu-radioisotope is provided as an aqueous solution with a radioactivity of between about 1,000 to about 100,000 MBq. In an embodiment, the radioactivity of the ⁶⁷Cu-radioisotope is about 1,000 MBq. In another embodiment, the radioactivity of the ⁶⁷Cu-radioisotope is about 5,000 MBq. In another embodiment, the radioactivity of the ⁶⁷Cu-radioisotope is about 10,000 MBq. In another embodiment, the radioactivity of the ⁶⁷Cu-radioisotope is about 15,000 MBq. In another embodiment, the radioactivity of the ⁶⁷Cu-radioisotope is about 20,000 MBq. In another embodiment, the radioactivity of the ⁶⁷Cu-radioisotope is about 25,000 MBq. In another embodiment, the radioactivity of the ⁶⁷Cu-radioisotope is about 30,000 MBq. In another embodiment, the radioactivity of the ⁶⁷Cu-radioisotope is about 35,000 MBq. In another embodiment, the radioactivity of the ⁶⁷Cu-radioisotope is about 40,000 MBq. In another embodiment, the radioactivity of the ⁶⁷Cu-radioisotope is about 45,000 MBq. In another embodiment, the radioactivity of the ⁶⁷Cu-radioisotope is about 50,000 MBq. In another embodiment, the radioactivity of the ⁶⁷Cu-radioisotope is about 55,000 MBq. In another embodiment, the radioactivity of the ⁶⁷Cu-radioisotope is about 60,000 MBq. In another embodiment, the radioactivity of the ⁶⁷Cu-radioisotope is about 65,000 MBq. In another embodiment, the radioactivity of the ⁶⁷Cu-radioisotope is about 70,000 MBq. In another embodiment, the radioactivity of the ⁶⁷Cu-radioisotope is about 75,000 MBq. In another embodiment, the radioactivity of the ⁶⁷Cu-radioisotope is about 80,000 MBq. In another embodiment, the radioactivity of the ⁶⁷Cu-radioisotope is about 85,000 MBq. In another embodiment, the radioactivity of the ⁶⁷Cu-radioisotope is about 90,000 MBq. In another embodiment, the radioactivity of the ⁶⁷Cu-radioisotope is about 95,000 MBq. In another embodiment, the radioactivity of the ⁶⁷Cu-radioisotope is about 100,000 MBq.

A mixture of a ⁶⁷Cu ion, a compound of Formula (I), or a salt thereof, and the sodium phosphate buffer solution comprising gentisic acid, or a salt thereof, may be allowed to stand at room temperature. The mixture may be allowed to stand with stirring, alternatively, the mixture is allowed to stand without stirring. The mixture may be allowed to stand for a time between about 5 to about 25 minutes. In an embodiment, the mixture of a ⁶⁷Cu ion, a compound of Formula (I), or a salt thereof, and the sodium phosphate buffer solution comprising gentisic acid, or a salt thereof is allowed to stand without stirring for about 5 minutes. In another embodiment, the mixture of a ⁶⁷Cu ion, a compound of Formula (I), or a salt thereof, and the sodium phosphate buffer solution comprising gentisic acid, or a salt thereof is allowed to stand without stirring for about 10 minutes. In another embodiment, the mixture of a ⁶⁷Cu ion, a compound of Formula (I), or a salt thereof, and the sodium phosphate buffer solution comprising gentisic acid, or a salt thereof is allowed to stand without stirring for about 15 minutes. In another embodiment, the mixture of a ⁶⁷Cu ion, a compound of Formula (I), or a salt thereof, and the sodium phosphate buffer solution comprising gentisic acid, or a salt thereof is allowed to stand without stirring for about 20 minutes. In another embodiment, the mixture of a ⁶⁷Cu ion, a compound of Formula (I), or a salt thereof, and the sodium phosphate buffer solution comprising gentisic acid, or a salt thereof is allowed to stand without stirring for about 25 minutes. In another embodiment, the mixture of a ⁶⁷Cu ion, a compound of Formula (I), or a salt thereof, and the sodium phosphate buffer solution comprising gentisic acid, or a salt thereof is allowed to stand without stirring for less than 25 minutes. In another preferred embodiment, the mixture of a ⁶⁷Cu-radioisotope, a compound of Formula (I), or a salt thereof, and the sodium phosphate buffer solution comprising gentisic acid, or a salt thereof is allowed to stand without stirring for about 25 minutes. In certain embodiments, the mixture of a ⁶⁷Cu ion and a compound of Formula (I) or a salt thereof is allowed to stand at room temperature, where room temperature is defined as ambient temperature in accordance with the USP. In certain embodiments, the ambient temperature is between about 15° C. and about 25° C.

According to another embodiment of the present invention, the mixture of a ⁶⁷Cu ion, a compound of Formula (I), or a salt thereof, and the sodium phosphate buffer solution comprising gentisic acid, or a salt thereof, is filtered. The mixture may be filtered through a solid phase extraction process. The mixture may be filtered through a solid phase extraction process, where the stationary phase of the solid phase extraction cartridge retains the compound of Formula (I), or a salt thereof, complexed with a Cu ion, any compound of Formula (I), or a salt thereof, that is not complexed and some gentisic acid in the form of a salt that is present, such as sodium gentisate. As used herein, the term “stationary phase” refers to a resin-like material that is held within the solid phase extraction cartridge and allows for the separation of compounds based on their polarity.

The solid phase extraction process as described herein may use a reverse-phase stationary phase. As used herein, the term “reverse-phase” in relation to a stationary phase refers to a stationary phase that is hydrophobic in nature, such that the stationary phase has an affinity for hydrophobic or uncharged molecules. Examples of a reverse-phase stationary phase may include Phenomenex Strata-X 33u Polymeric Reversed Phase, Waters tC18 or Waters C18. Other similar stationary phases may be used. As the solid phase extraction process uses a reverse-phase stationary phase, any free ⁶⁷Cu ions and the remaining gentisic acid or its salt is not retained by the stationary phase and these components are discarded.

In an embodiment, the mixture of a ⁶⁷Cu ion, a compound of Formula (I) and the sodium phosphate buffer solution comprising gentisic acid, or a salt thereof is filtered through a solid phase extraction cartridge. In an embodiment, the mixture of a Cu ion, a compound of Formula (I) and the sodium phosphate buffer solution comprising gentisic acid, or a salt thereof, is filtered through a solid phase extraction cartridge with a reverse-phase stationary phase. In an embodiment, the compound of Formula (I) complexed with a ⁶⁷Cu ion is retained by a solid phase extraction cartridge with a reverse-phase stationary phase. In a preferred embodiment, the mixture of a ⁶⁷Cu-radioisotope, a compound of Formula (I) and the sodium phosphate buffer solution comprising gentisic acid, or a salt thereof is filtered through a solid phase extraction cartridge with reverse-phase stationary phase. In another preferred embodiment, the compound of Formula (I) complexed with a ⁶⁷Cu ion is retained by a solid phase extraction cartridge with a reverse-phase stationary phase.

The compound of Formula (I) complexed with a ⁶⁷Cu ion is eluted from the solid phase extraction cartridge containing the stationary phase by washing with a solvent. As the solid phase extraction cartridge contains a reverse-phase stationary phase, eluting the compound of Formula (I) complexed with a ⁶⁷Cu ion requires washing of the stationary phase with ethanol, saline and/or another solvent. In an embodiment, the solid phase extraction cartridge is washed with ethanol to elute the compound of Formula (I) complexed with a ⁶⁷Cu ion. In another embodiment, the solid phase extraction cartridge is washed with saline to elute the compound of Formula (I) complexed with a ⁶⁷Cu ion. In another embodiment, the solid phase extraction cartridge is washed with ethanol and saline to elute the compound of Formula (I) complexed with a ⁶⁷Cu ion. In a preferred embodiment, the solid phase extraction cartridge is washed with ethanol and comprising ascorbic acid to elute the compound of Formula (I) complexed with a ⁶⁷Cu ion. In a preferred embodiment, the solid phase extraction cartridge is washed with ethanol comprising ascorbic acid to provide the formulation of the present invention.

A person skilled in the art would understand that the excipients of the formulation include the solvent that is used to elute the compound of Formula (I) complexed with a ⁶⁷Cu ion from the stationary phase, and that the amount of each solvent used is related to the amount of each excipient in the formulations of the present invention.

A person skilled in the art would understand that the present disclosure provides a manual process for producing a formulation according to the present invention. A person skilled in the art would understand that the steps described herein may be automated, by using a suitable automated radiosynthesis module, in order to obtain a formulation according to the present invention.

The present inventors have found that the formulations disclosed herein have greater stability and show reduced radiolysis in light of the higher starting radioactivity. This enhanced stability may be attributed to the increased radiochemical purity of the formulation at a given radioactivity. The stability of the formulations of the present invention may be observed for a time of up to 96 hours post-manufacture. Where the formulations of the present invention are used for the purposes of treatment or therapy, the greater stability may mean that doses for multiple patients at multiple remote locations can be prepared at the same time at a single facility. This may mean that resources for manufacture are required at a single facility, rather than at multiple facilities, and greater efficiency in production of the formulations may be achieved. Where the formulations of the present invention are used for imaging purposes, further advantages may be provided since the clinical imaging sites can receive a dosage form that is ready to inject. This may be particularly advantageous for clinical sites where dedicated radiopharmaceutical production facilities do not exist.

The formulations of the present invention comprise a ligand-radioisotope complex, where the ligand is a compound of Formula (I), or a salt thereof. The compound of Formula (I), or a salt thereof, and the radioisotope may be supplied in separate containers. Alternatively, the compound of Formula (I), or a salt thereof, and the radioisotope may be supplied together as a ligand-radioisotope complex.

The container consisting of the compound of Formula (I), or a salt thereof, may provide the compound of Formula (I), or a salt thereof, as a lyophilised powder. The container may be provided at a temperature of between −20° C. and 20° C.

The formulations may be provided as a kit comprising a container of the ⁶⁷Cu radioisotope and a separate container with the ligand and instructions for making the aqueous formulation of the present invention. In an embodiment, the kit of the present invention comprises a container providing a solution of a ⁶⁷Cu radioisotope and a separate container providing a compound of Formula (I), or a salt thereof. The container providing the radioisotope may contain a solution of a ⁶⁷Cu salt.

In an embodiment, a kit of the present invention comprises a container with a solution of ⁶⁷Cu radioisotope. In another embodiment, a kit of the present invention comprises a container with a solution of a copper salt containing a ⁶⁷Cu radioisotope. In another embodiment, a kit of the present invention comprises a container with a solution of a chloride salt containing a ⁶⁷Cu radioisotope. In another embodiment, a kit of the present invention comprises a container with a solution of a radioactive copper(II) chloride salt. In another embodiment, a kit of the present invention comprises a container with a solution of a copper(II) chloride salt, wherein the copper ion is the ⁶⁷Cu isotope. In another embodiment, a kit of the present invention comprises a container with a solution of [⁶⁷Cu]CuCl₂.

The solution of the radioisotope is typically provided as an aqueous solution. In an embodiment, a kit of the present invention provides a radioisotope in the form of an aqueous solution. In a further embodiment, a kit of the present invention provides a radioisotope in the form of an acidic aqueous solution. In another embodiment, a kit of the present invention provides a radioisotope as a solution in hydrochloric acid. The radioisotope may be provided as a solution in hydrochloric acid at a concentration of between about 0.01 and about 0.1 mol/L.

In an embodiment, a kit of the present invention comprises a container with a solution of [⁶⁷Cu]CuCl₂ in hydrochloric acid. In another embodiment, a kit of the present invention comprises a container with a solution of [⁶⁷Cu]CuCl₂ in hydrochloric acid, wherein the hydrochloric acid is at a concentration of about 0.02 mol/L. In another embodiment, a kit of the present invention comprises a container with a solution of [⁶⁷Cu]CuCl₂ in hydrochloric acid, wherein the hydrochloric acid is at a concentration of about 0.05 mol/L. In another embodiment, a kit of the present invention comprises a container with a solution of [⁶⁷Cu]CuCl₂ in hydrochloric acid, wherein the hydrochloric acid is at a concentration of about 0.1 mol/L.

The kit may further comprise a container consisting of sodium phosphate buffer, ethanol, gentisic acid, or a salt thereof and ascorbic acid, or a salt thereof. The kit may comprise a container consisting of sodium phosphate buffer and gentisic acid in an aqueous solution and a second container consisting of a solution of aqueous ethanol and ascorbic acid, or a salt thereof or alternatively, the container may consist only of ethanol, ascorbic acid, or a salt thereof and gentisic acid, or a salt thereof. In an embodiment, the kit comprises a container comprising sodium phosphate buffer and gentisic acid, or a salt thereof and a second container comprising aqueous ethanol and ascorbic acid, or a salt thereof.

### Uses of a Formulation of the Present Invention

Formulations of the present invention may be particularly useful for the purposes of diagnosis and treatment in medicine. Complexes with a ligand bearing an appropriate targeting fragment can be used to locate specific tissue types. For such complexes to be considered suitable for use in in vivo diagnosis and treatment, the complex must display appropriate kinetic, stability and clearance properties under physiological conditions, in addition to the requisite solubility and stability properties of the complex in solution. As used herein, the term “complex” may relate to a ligand-metal ion complex, where the metal ion is a radioactive isotope or alternatively, the metal ion is a non-radioactive isotope.

The present inventors have found that the formulations of the present invention comprising a compound of Formula (I), or a salt thereof, containing a sarcophagine, a bombesin-like peptide, the propylamide linker and the linker comprising a PEG group shows affinity for the GRP receptor. The combination of each of these components in the compound of Formula (I) allow for administration of the corresponding complex containing a radionuclide, maintaining stability of the complex in vivo and accumulation of the complex at the intended target.

The formulations of the present invention comprising a compound of Formula (I) complexed with a ⁶⁷Cu radionuclide may be used in methods of radioimaging, diagnosis or therapy. Accordingly, the present invention provides a method for radioimaging, a method for diagnosing a disease in a subject or a method for therapy of a disease in a subject, comprising administering to the subject an effective amount of a formulation as defined herein. The present inventors have found that the formulations of the present invention may be used in a method for radioimaging, a method for diagnosing or a method for therapy of a cancer.

Radioimaging of a subject to which a ⁶⁷Cu-radiolabeled compound of Formula (I) is administered may be by positron emission tomography (PET) or by single-photon emission computed tomography (SPECT). In an embodiment, the present invention provides a method for radioimaging a subject in need thereof, the method comprising administering a formulation of the present invention comprising a compound of Formula (I), or a salt thereof, complexed with a ⁶⁷Cu radionuclide. In certain embodiments, the present invention relates to a method for the treatment of a cancer in a subject in need thereof, the method comprising the step of administering an aqueous formulation comprising a compound of Formula (I), or a salt thereof, complexed with a ⁶⁷Cu ion:

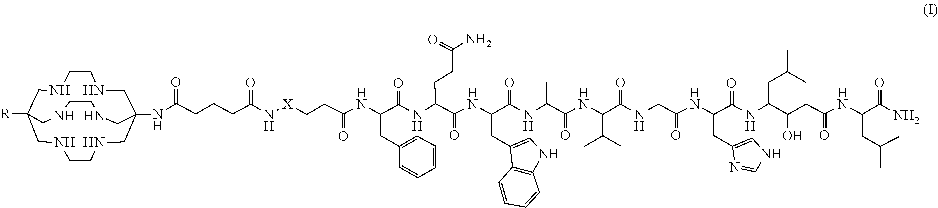

wherein:
X is

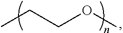

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A):

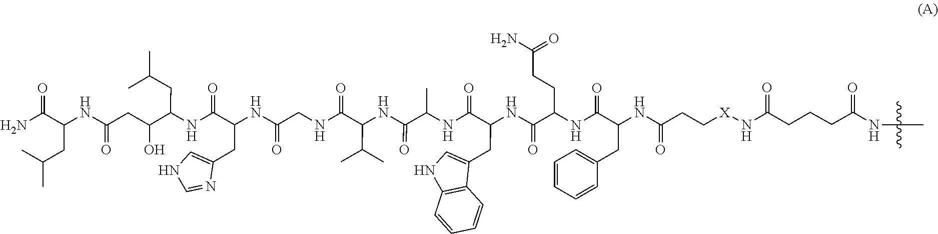

the formulation further comprising a buffer and:
about 0.01% to about 0.1% (w/v) gentisic acid or a salt thereof;
about 1% to about 7% (v/v) ethanol; and
about 4% to about 10% (w/v) ascorbic acid or a salt thereof.

In certain embodiments of the methods disclosed herein, R is a group of the formula (A):

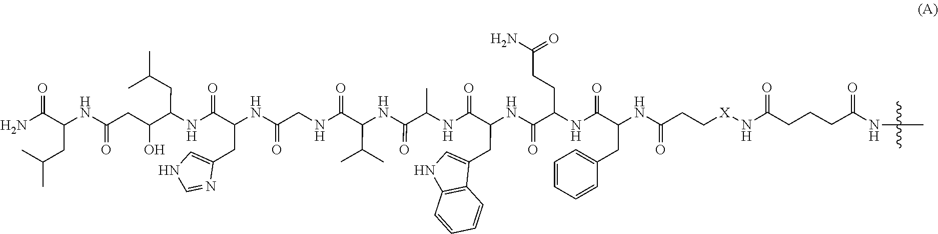

wherein X is as defined above, such as with the below stereochemistry:

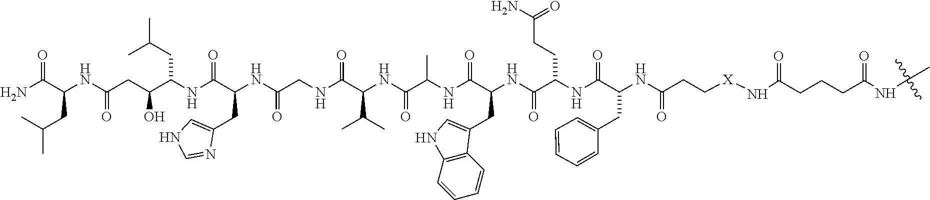

In certain embodiments of the methods disclosed herein, R is an optionally substituted C₁-C₁₂ alkyl.

In a further embodiment, R is methyl.

In a further embodiment of the methods disclosed herein, X is

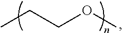

wherein n is an integer from 1 to 10.

In a further embodiment, X is

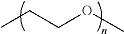

and n is 4.

In certain embodiments, the methods for the treatment of a cancer further comprise the step of radioimaging of the subject. In certain embodiments, radioimaging of the subject is performed about 12 hours after the aqueous formulation is administered. In other embodiments, radioimaging of the subject is performed about 24 hours after the aqueous formulation is administered. In other embodiments, radioimaging of the subject is performed about 36 hours after the aqueous formulation is administered. In other embodiments, radioimaging of the subject is performed about 48 hours after the aqueous formulation is administered.

In an embodiment, radioimaging of the subject after administration of the compound of Formula (I), or a salt thereof, complexed by a radionuclide is by PET. In another embodiment, radioimaging of the subject after administration of the compound of Formula (I), or a salt thereof, complexed by a radionuclide is by SPECT.

The formulations of the present invention may be administered to a subject in need thereof as a composition by a parenteral route. Administration by intravenous injection may be preferred. Alternatively, the formulations of the present invention may be given by intraarterial or other routes, for delivery into the systemic circulation. The subject to which the compound is administered is then placed into a PET (or SPECT) scanner and images showing the localisation of the complex, and subsequently location of any cancers or tumours, are obtained. This then allows for diagnosis and detection of a cancer or tumour.

The formulations of the present invention may be used in methods of treatment of diseases, such as cancers. The methods disclosed herein comprise administration of an effective amount of a formulation as defined herein comprising compound of Formula (I), or a salt thereof, complexed with a ⁶⁷Cu radionuclide to a subject in need thereof. The compound contains a bombesin-like peptide, which binds at GRP receptors that are expressed at sites of various cancers. Given the abundance of such receptors are associated with particular types of cancer, the accumulation of compounds of the present invention as detected by the radioactive decay of the radionuclide indicates the location of the cancer. The present inventors have found that compounds of the present invention show a particular affinity for the GRP receptor. Furthermore, the presence of both the propylamide linker and the linker comprising a PEG group contribute to provide a complex (when the compound is radiolabeled with a radionuclide) that is capable of administration to a subject and subsequent localization at sites overexpressing the GRP receptor. The formulations of the present invention comprising a compound of Formula (I), or a salt thereof, also have the requisite stability with respect to the ⁶⁷Cu radionuclide. For example, the sarcophagine present in the compound is capable of chelating ⁶⁷Cu such that the ⁶⁷Cu remains coordinated upon administration to a subject and subsequent binding at the target site. Since the ⁶⁷Cu remains coordinated and localized to the target site due to binding of the compound as a whole, radiation damage at other sites (e.g. healthy tissue) is minimized.

As used herein the term “cancer” broadly encompasses a class of neoplastic diseases characterised by abnormal cell growth with the potential to invade or spread to other parts of the body. In one embodiment, the cancer is one that expresses or overexpresses the GRP receptor. These are to be contrasted with benign tumours, which do not spread to other parts of the body and therefore the definition as used herein includes all malignant (cancerous) disease states. The term therefore encompasses the treatment of tumours.

Accordingly, the term “tumour” is used generally to define any malignant cancerous or pre-cancerous cell growth, and may include blood based cancers, but is particularly directed to solid tumours or carcinomas such as prostate cancer, breast cancer, gliomas, gastrointestinal stromal tumours, melanomas, colon, lung, ovarian, skin, pancreas, pharynx, brain, CNS, and renal cancers (as well as other cancers). In some embodiments, the tumour is associated with a cancer selected from the group consisting of prostate cancer, breast cancer, ovarian cancer, urinary cancer, small cell lung cancers, glioblastoma and gastrointestinal stromal tumours.

In order for the complex to be suitable for radioimaging purposes, the radioisotope-ligand complex must display sufficient metabolic stability, i.e. that the complex remains intact with the radioisotope bound to the ligand, for a requisite time. The present invention provides a complex of a compound of Formula (I), or a salt thereof, and ⁶⁷Cu that remains intact for up to 96 hours, as evidenced by the absence of radioisotope loss and metabolic decomposition.

In an embodiment, the present invention provides the use of a formulation comprising a compound of Formula (I), or a salt thereof, complexed with ⁶⁷Cu in a method for the radioimaging of a tumour or cancer. One skilled in the art would understand that the information obtained from radioimaging of a subject may be used in the diagnosis of a tumour or cancer in the subject. In an embodiment, the present invention provides a method for the diagnosis of a tumour or cancer. In a further embodiment, the tumour or cancer may be a GRP receptor expressing tumour or cancer. In an embodiment, the tumour or cancer is a prostate cancer. In another embodiment, the tumour or cancer is a breast cancer. In another embodiment, tumour or cancer is a glioma. In another embodiment, the tumour or cancer is a gastrointestinal stromal tumour. In another embodiment, the tumour or cancer is a brain cancer. In another embodiment, the tumour or cancer is a melanoma. In another embodiment, the tumour or cancer is a lung cancer. In another embodiment, the tumour or cancer is a colon cancer. In another embodiment, the tumour is a pancreatic tumour. In another embodiment, the tumour is a renal tumour.

The administration of the formulation may treat a tumour or cancer. As discussed above, the compound of Formula (I), or salt thereof, may bind GRP receptors on the surface of a tumour or cancer site, such the binding of the compound to locations with GRP receptors also brings the ⁶⁷Cu radioisotope into close proximity of this location. As the ⁶⁷Cu radioisotope undergoes radioactive decay, with the mode of decay dependent on the exact radioisotope chosen, the products of decay may be useful in the treatment of a tumour or cancer due to the proximity of the tumour or cancer to the compound of Formula (I), or salt thereof, and ⁶⁷Cu radioisotope.

In an embodiment, the present invention provides the use of a formulation comprising a compound of Formula (I), or a salt thereof, complexed with ⁶⁷Cu in a method for treatment of a tumour or cancer. In an embodiment, the tumour or cancer may be a GRP receptor expressing tumour or cancer. In an embodiment, the tumour or cancer is a prostate cancer. In another embodiment, the tumour or cancer is a breast cancer. In another embodiment, tumour or cancer is a glioma. In another embodiment, the tumour or cancer is a gastrointestinal stromal tumour. In another embodiment, the tumour or cancer is a brain cancer. In another embodiment, the tumour or cancer is a melanoma. In another embodiment, the tumour or cancer is a lung cancer. In another embodiment, the tumour or cancer is a colon cancer. In another embodiment, the tumour is a pancreatic tumour. In another embodiment, the tumour is a renal tumour.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

Phosphate buffers for radiolabelling were prepared using sodium phosphate dibasic (anhydrous), sodium phosphate monobasic and TraceSELECT water. All buffers were stored at room temperature while not in use.

Copper-67 (⁶⁷Cu) was obtained from NorthStar Medical Radioisotopes as [⁶⁷Cu]CuCl₂ as a dried powder and reconstituted in TraceSELECT water, with a starting activity of 13.68 GBq in a total volume of 0.24 mL.

Analysis of the formulations was performed by radioHPLC and TLC. HPLC was performed with mobile phases of 0.1% trifluoroacetic acid in water (A) and 0.1% trifluoroacetic acid in acetonitrile (B) using a gradient of 15% B in A to 39% B in A over 12 min using a Ascentis Express C18, 2.7 μm, 90 Å, 150 mm×4.6 mm HPLC column.

TLC was performed with a mobile phase of 10 mM NaEDTA in 50 mM phosphate buffer using a silica gel 60 F₂₅₄ aluminium backed TLC plate with dimensions of 1 cm×10 cm.

MeCN for HPLC (Honeywell, Lot #S1RA1H) and trifluoroacetic acid for HPLC (TFA, ReagentPlus, 99%, Sigma Aldrich), (+)-Sodium L-ascorbate (Sigma Aldrich, >99%, Lot #: BCBV4424), and gentisic acid sodium salt hydrate (Sigma Aldrich, >99%, Lot #: MKCC2280) were used as received. All HPLC mobile phases were prepared prior to use, filtered (using a 0.45 μm aqueous or organic filter) and degassed via a combination of ultrasonic irradiation under vacuum for 10 minutes. All ethanol used was 200 Proof USP ethanol. All syringes used were ‘B Braun Injekt-F’.

The compound of Formula (I), i.e. Sar-BBN, was obtained from Auspep Clinical Peptides, Tullarmine VIC, Australia, with a purity of ≥95%.

### Example 1—Preparation of a Single Dose Formulation Comprising a Compound of Formula (Ib) Complexed with ⁶⁷Cu

Sodium gentisate (10 mg) is dissolved in 0.1 M sodium phosphate buffer solution (14 mL) to provide a first solution (Solution A). 120 g of the compound of Formula (Ia) is then dissolved in Solution A (14 mL) to provide a Reaction Vial.

The radioactivity of a ⁶⁷Cu chloride solution is measured and the time recorded. The ⁶⁷Cu chloride solution is then added into the Reaction Vial containing the compound of Formula (Ia) in solution and is held at ambient temperature for 25 minutes.

A second solution (Solution B) is prepared by dissolving sodium ascorbate (2 g) in TraceSELECT water (12 mL) and ethanol (1.1 mL).

The contents of the Reaction Vial are then transferred via a sterile filter into a Final Product Vial. Solution B is drawn up (4 mL) in a 10 mL syringe and used to rinse out the Reaction Vial. The contents of the Reaction Vial are transferred via the sterile filter into the Final Product Vial before gently homogenising the contents. The activity within the Final Product Vial is assayed and the EOS time and final product volume are recorded. Total final volume of ˜27 mL after QC sampling.

Table 1 below reproduces a quality control test summary for an aqueous formulation comprising [⁶⁷Cu]Formula (Ib) prepared according to the above method.

TABLE 1
Quality control test summary for an aqueous
formulation comprising [⁶⁷Cu]Formula (Ib)

| QC Test Performed | Acceptance Criteria | Batch QC Results | Pass | Fail |
|---|---|---|---|---|
| Appearance/Visible | Final product is clear, | Clear ✓ | ✓ | |
| particles in injection | colourless to pale yellow, | Colourless to | | |
| | and free of visible | pale yellow ✓ | | |
| | particulates | Particle-free ✓ | | |
| pH by probe | 4.0-8.0 | 6.72 | ✓ | |
| Free [⁶⁷Cu]Cu²⁺ by TLC | Free [⁶⁷Cu]Cu²⁺ ≤5% | 0% | | |
| Radiochemical purity by | RCP by radio-HPLC ≥90% | 94.23% | ✓ | |
| radio-HPLC | | | | |
| Radiochemical identity by | RT of [⁶⁷Cu]Formula (Ib) | 0% | ✓ | |
| radio-HPLC | sample is within ±5% of | | | |
| | RT of reference std. | | | |
| Radionuclide identity by | Peaks should be identified | Consistent with | ✓ | |
| Gamma Spec | at 511 and 1346 keV | ⁶⁷Cu | | |

### Stability of Formulations of the Invention

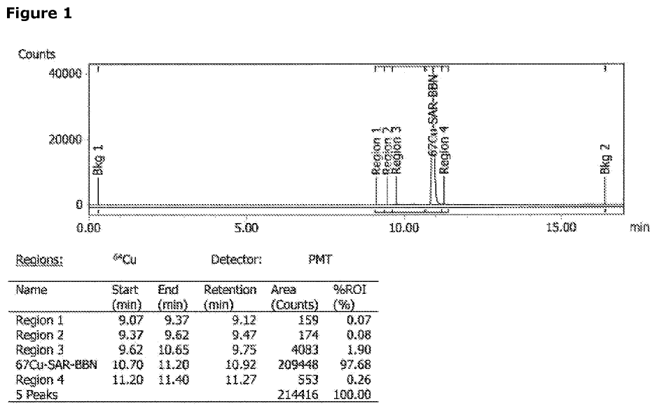

Product stability was monitored up to 96 hours post End of Synthesis (EOS) for 3 validation batches of [⁶⁷Cu]Formula(Ib) in an aqueous formulation prepared as outlined above. Over the period of the test, the radiochemical purity (RPC) did not fall below 94.0%. FIG. 1 illustrates the radio-HPLC chromatograph at 96 hours post EOS.

1. An aqueous formulation comprising a compound of Formula (I), or a salt thereof, complexed with a ⁶⁷Cu ion: 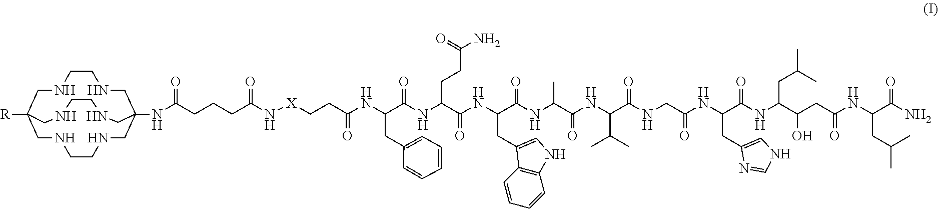
wherein:
X is 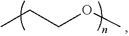
where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A): 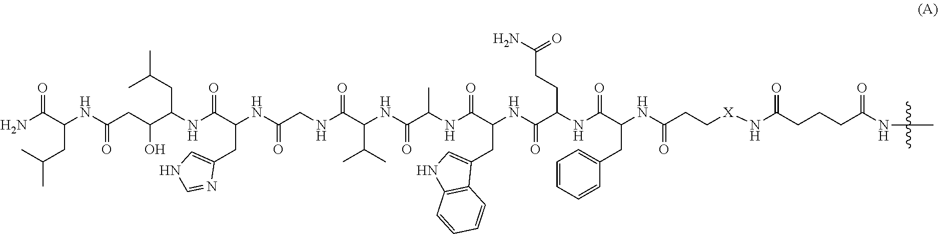
the formulation further comprising a buffer and:
about 0.01% to about 0.1% (w/v) gentisic acid or a salt thereof;
about 1% to about 7% (v/v) ethanol; and
about 4% to about 10% (w/v) ascorbic acid or a salt thereof.

2. The aqueous formulation according to claim 1, wherein for the compound of Formula (I) or the salt thereof, R is a group of the formula (A): 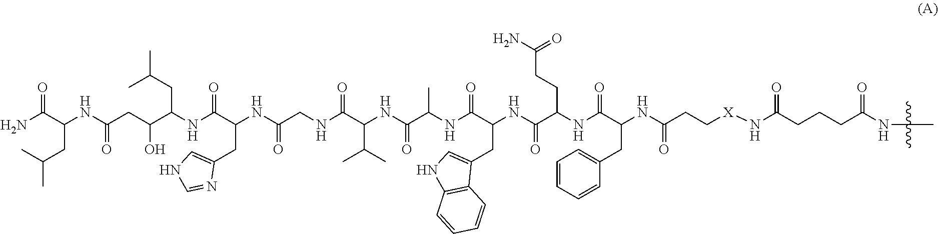
wherein X is as defined in claim 1.

3. The aqueous formulation according to claim 1, wherein the group of formula (A) has the below stereochemistry:

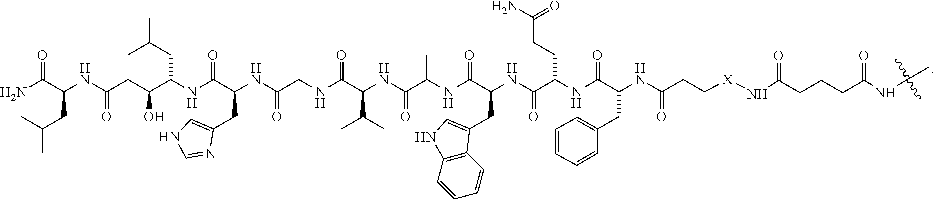

4. (canceled)

5. The aqueous formulation according to claim 1, wherein R is methyl.

6. The aqueous formulation according to claim 1, wherein for the compound of Formula (I) or the salt thereof, X is 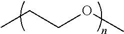
wherein n is an integer from 1 to 10.

7. (canceled)

8. The aqueous formulation according to claim 1, wherein the compound of Formula (I), or the salt thereof, is selected from the Formula (Ia): 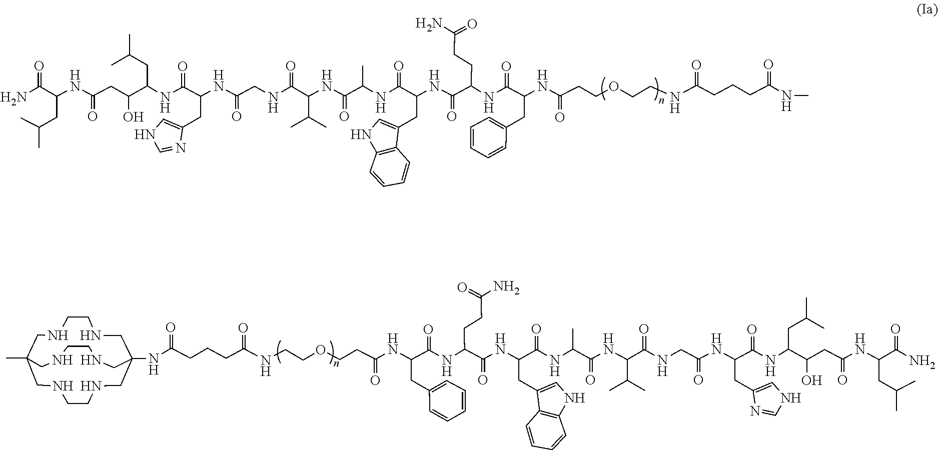
or the Formula (Ib):

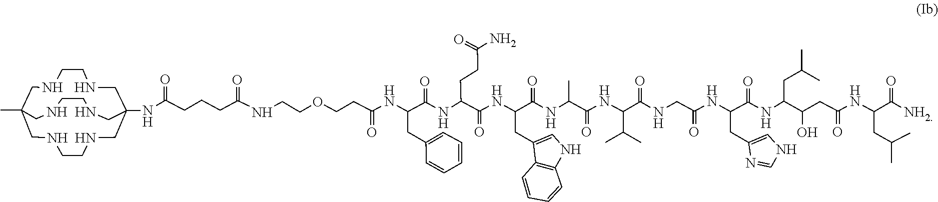 [SMILES: CC(C)CC(NC(=O)CC(O)C(CC(C)C)NC(=O)C(Cc1c[nH]cn1)NC(=O)CNC(=O)C(NC(=O)C(C)NC(=O)C(Cc1c[nH]c2ccccc12)NC(=O)C(CCC(N)=O)NC(=O)C(Cc1ccccc1)NC(=O)CCOCCNC(=O)CCCC(=O)NC12CNCCNCC(C)(CNCCNC1)CNCCNC2)C(C)C)C(N)=O]

9. The aqueous formulation according to claim 1, wherein the buffer is sodium phosphate buffer.

10. (canceled)

11. The aqueous formulation according to claim 1, wherein the gentisic acid salt is sodium gentisate.

12. (canceled)

13. The aqueous formulation according to claim 1, wherein the formulation has a pH of about 4 to about 8.

14. The aqueous formulation according to claim 1, wherein the formulation has a radiochemical purity of more than about 90%, 92%, 94%, 96% or 98% for a time of at least 96 hours; or
wherein the amount of free ⁶⁷Cu in the formulation is no more than about 5%, 4%, 3%, 2% or 1% of the total amount of ⁶⁷Cu present.

15. (canceled)

16. The aqueous formulation according to claim 1, wherein the radioactive concentration of the formulation is about 0.1 GBq/mL to about 1.0 GBq/mL.

17. A process for preparing an aqueous formulation comprising a compound of Formula (I) or a salt thereof complexed with a ⁶⁷Cu ion, the process comprising the steps of:
i) dissolving a compound of Formula (I), or a salt thereof, in a buffer solution comprising gentisic acid, or a salt thereof;
ii) adding a solution of a ⁶⁷Cu ion to the solution of step i);
iii) filtering the solution obtained from step ii); and
iv) diluting the reaction by addition of aqueous ethanol and ascorbic acid;
to recover an aqueous formulation comprising a compound of Formula (I), or a salt thereof, complexed with a ⁶⁷Cu ion.

18. The process according to claim 17, wherein the buffer solution is sodium phosphate buffer.

19. The process according to claim 17, wherein the gentisic acid or the salt thereof, is present in an amount of about 0.01% to about 0.1% (w/v), the aqueous ethanol is present in an amount of about 1% to about 7% (v/v) and the ascorbic acid or a salt thereof is present in an amount of about 4% to about 10% (w/v), or
wherein the gentisic acid or the salt thereof, is present in an amount of about 0.03% to about 0.04% (w/v), the aqueous ethanol is present in an amount of about 4% (v/v), and the ascorbic acid or a salt thereof is present in an amount of about 6.5% to about 8% (w/v).

20. (canceled)

21. An aqueous formulation prepared by the process according to claim 17.

22. A kit for making an aqueous formulation comprising a compound of Formula (I), or a salt thereof, complexed with a ⁶⁷Cu ion, the kit comprising:
a container comprising a lyophilised compound of Formula (I), or a salt thereof: 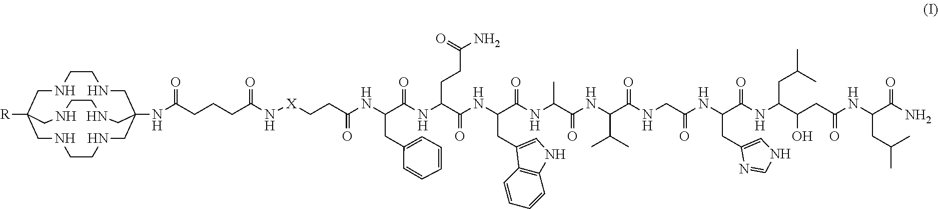
wherein:
X is 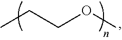
where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A): 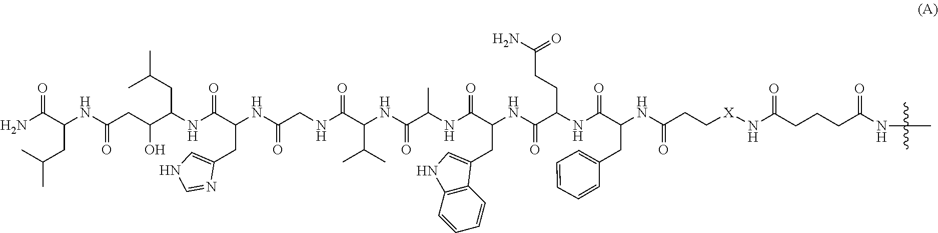
a container comprising a solution of a ⁶⁷Cu ion; and
instructions for preparing an aqueous formulation, including the addition of sodium phosphate buffer, ethanol, gentisic acid, or a salt thereof and ascorbic acid, or a salt thereof.

23. (canceled)

24. A method for radioimaging, diagnosing or treating a cancer, the method comprising administering to a subject in need thereof an aqueous formulation according to claim 1, wherein the cancer is associated with expression of a GRP receptor.

25. (canceled)

26. The method according to claim 24, wherein the cancer is selected from the group consisting of prostate cancer, breast cancer, ovarian cancer, urinary cancer, small cell lung cancers, glioblastoma and gastrointestinal stromal tumours.

27. The method according to claim 24, wherein the formulation administered contains a dose of about 1 GBq to about 20 GBq of radioactivity.

28. The method according to claim 24, wherein the method is for the treatment of a cancer and the method further comprises the step of radioimaging of the subject.
